(19) United States
(12) Patent Application Publication
(10) Pub. No.: US 2026/0268460 A1
(43) Pub. Date: Sep. 10, 2026

(54) SYSTEMS AND METHODS FOR IMPROVING IMAGE QUALITY THROUGH AN ENDOSCOPE

(71) Applicant: VOPE MEDICAL INC., Montreal (CA)

(72) Inventors: Timothée BERNARD, Montreal (CA); Sukesh ADIGA VASUDEVA, Montreal (CA); Amy LORINCZ, Montreal (CA)

(21) Appl. No.: 19/546,004

(22) Filed: Feb. 20, 2026

Related U.S. Application Data
(63) Continuation of application No. PCT/CA2025/050190, filed on Feb. 13, 2025.
(60) Provisional application No. 63/554,430, filed on Feb. 16, 2024.

(51) Int. Cl. G06T 5/77; G06T 3/40; G06T 5/50; G06T 5/60; G06T 7/00

(52) U.S. Cl. CPC G06T 5/77; G06T 3/40; G06T 5/50; G06T 5/60; G06T 7/0012; G06T 2207/10068; G06T 2207/20081; G06T 2207/20084; G06T 2207/30168

(57) ABSTRACT

A method comprising accessing a first image from a stream of images; determining that a contaminated segment is present within the first image; determining at least one of a type of contaminant associated with the contaminated segment or a dimension of the contaminated segment; adjusting at least one of a resolution of images or a frame rate associated with the stream of images, the adjusting being based on the at least one of the type of contaminant or the dimension of the contaminated segment, the adjusting resulting in at least one of generating reduced-resolution images or a reduced stream of images; inputting, to a first machine-learning algorithm (MLA), at least one reduced-resolution image or at least one extracted image taken from the reduced stream of images to generate a replacement segment for the contaminated segment; and generating a restored image based on the replacement segment.

### CROSS-REFERENCE

The present application is a continuation of PCT/CA2025/050190, filed Feb. 13, 2025, which claims priority on U.S. Provisional Patent Application No. 63/554,430, entitled “SYSTEMS AND METHODS FOR IMPROVING IMAGE QUALITY THROUGH AN ENDOSCOPE”, filed on Feb. 16, 2024, the contents of each of which are incorporated herein by reference in their entirety.

### FIELD

Various embodiments are described herein that generally relate to systems and methods for improving image quality through an endoscope. More particularly, the present invention relates to restoring an endoscopic video signal in real time.

### INTRODUCTION

The following paragraphs are provided by way of background to the present disclosure. They are not, however, an admission that anything discussed therein is prior art or part of the knowledge of persons skilled in the art.

Unclear vision while operating is a problem that surgeons experience in minimally invasive surgery (MIS), a kind of surgery introducing instruments into the patient, as opposed to open surgery. One of these instruments is an endoscopic camera which can be obstructed by different types of contaminants such as blood, fog, blur, fluids, etc.

There is need to reduce, mitigate, or eliminate unclear vision in MIS. For example, 26% more errors in surgery occur when stressed. Fifteen million MIS procedures are performed annually worldwide. As well, 37% of the time, the surgical site view is obstructed due to contamination.

The invention addresses a major issue in minimally invasive surgery, a type of surgery involving the introductions of cameras and instruments inside the body through small incisions as opposed to “open surgery”. During minimally invasive surgeries, the view is obstructed 37% of the time by contaminants such as blood, tissues, condensation in the form of fog, and smoke. According to Arora, Sonal, et al. “The Impact of Stress on Surgical Performance: A Systematic Review of the Literature.” Surgery, vol. 147, no. 3, 2010, doi:10.1016/j.surg.2009.10.007, visual impairment is the number one stress factor during surgery leading to increased error rates and longer operation time. More than 200 million endoscopic procedures are performed annually which makes it one of the most urgent problems to address in surgery.

The problem is a well-known concern in minimally invasive surgery. In the cleaning system landscape, we find multiple approaches for in-vivo cleaning such as mechanical systems, irrigative devices or energy-based, but none are able to penetrate the market due to multiple issues such as lack of compatibility, lack of performance and most of the time, very specific to one type of contaminant. The gold standard remains removing the scope from the body and cleaning it manually, making every cleaning event very disruptive during surgeries.

There is a clear need for improved systems and methods that address the challenges or shortcomings described above.

### SUMMARY

Various embodiments of a system and method for improving vision quality through an endoscope, and computer products for use therewith, are provided according to the teachings herein.

In a first broad aspect of the present technology there is provided a method for near real-time image enhancement of endoscopic images, the method executable by at least one processor, the method comprising: accessing a first image from a stream of images; determining that a contaminated segment is present within the first image; determining at least one of (i) a type of contaminant associated with the contaminated segment or (ii) a dimension of the contaminated segment; adjusting at least one of (iii) a resolution of images from the stream of images or (iv) a frame rate associated with the stream of images, the adjusting being based on the at least one of (v) the type of contaminant or (vi) the dimension of the contaminated segment, the adjusting resulting in at least one of (vii) generating reduced-resolution images from the stream of images or (viii) a reduced stream of images; inputting, to a first machine-learning algorithm (MLA), (ix) at least one reduced-resolution image from the reduced-resolution images or (x) at least one extracted image taken from the reduced stream of images to generate a replacement segment for the contaminated segment; and generating a restored image based on the replacement segment.

In some embodiments of the method, the contamination type is indicative of at least one of smoke, fog, blood, fat, blur, or presence of a surgical tool.

In some embodiments of the method, the method further comprises: accessing a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image ; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the method, the method further comprises: determining whether the first image is adequate for caching, the adequacy being determined based on at least one image quality metric; and in response to determining that the first image is adequate for caching, storing the first image in the cache memory as one of the plurality of cached images.

In some embodiments of the method, the at least one image quality metric comprises one or more of: a sharpness metric, determined based on at least one of an edge gradient value and a variance of the Laplacian; or a haze level metric, determined based on at least one of a Dark Channel Prior (DCP) value and a Fog Aware Density Evaluation (FADE) value; or an information richness metric, determined based on entropy analysis.

In some embodiments of the method, an individual cached image is removed from the cache memory in response to at least one of: the temporal relevance score of the individual cached image failing to exceed the temporal relevance threshold; and the contextual relevance score of the individual cached image failing to exceed the contextual relevance threshold.

In some embodiments of the method, the method further comprises selectively upscaling the restored image.

In some embodiments of the method, the method further comprises selectively increasing the frame rate associated with the stream of images.

In some embodiments of the method, the method further comprises selectively adjusting the dimension of the region of interest (ROI) used to determine that the contaminated segment is present within the first image.

In some embodiments of the method, the method further comprises: generating a visual cue on the restored image, the visual cue indicating at least one of: a region corresponding to the replacement segment; and a reliability score for the replacement segment within the composed image frame.

In some embodiments of the method, determining that the contaminated segment is present within the first image comprises executing a second machine-learning algorithm (MLA) by inputting the first image to the second MLA and outputting, by the second MLA, at least one of (i) a type of contaminant associated with the contaminated segment or (ii) a dimension of the contaminated segment.

In some embodiments of the method, prior to determining that the contaminated segment is present within the first image, the method executes grouping the first image with another image from the stream of images.

In some embodiments of the method, determining that the contaminated segment is present is executed on some images but not all images of the stream of images.

In some embodiments of the method, adjusting at least one of (iii) the resolution of images from the stream of images or (iv) the frame rate associated with the stream of images is based on available computing resources of a device on which the method is executed.

In some embodiments of the method, prior to inputting to the first MLA, (ix) the at least one reduced-resolution image from the reduced-resolution images or (x) the at least one extracted image taken from the reduced stream of images, the method executes: selecting the first MLA from a plurality of MLAs based on at least one of: the type of contaminant associated with the contaminated segment; or available computing resources of a device on which the method is executed.

In some embodiments of the method, the at least one extracted image taken from the reduced stream of images is further processed to reduce its resolution before being used to generate the replacement segment for the contaminated segment.

In some embodiments of the method, the method further comprises displaying the restored image.

In another broad aspect of the present technology, there is provided a method for near real-time image enhancement of endoscopic images, the method executable by at least one processor, the method comprising: accessing a first image from a stream of images; determining that a contaminated segment is present within the first image; determining at least one of (i) a type of contaminant associated with the contaminated segment or (ii) a dimension of the contaminated segment; adjusting at least one of (iii) a resolution of the first image or (iv) a frame rate associated with the stream of images, the adjusting being based on the at least one of (v) the type of contaminant or (vi) the dimension of the contaminated segment, the adjusting resulting in at least one of (vii) generating a reduced-resolution image from the first image or (viii) a reduced stream of images; inputting, to a machine-learning algorithm (MLA), (ix) the reduced-resolution image or (x) at least one extracted image taken from the stream of reduced images to generate a replacement segment for the contaminated segment; and generating a restored image based on the replacement segment.

In some embodiments of the method, the method further comprises: accessing a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image ; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the method, the method further comprises: displaying the restored image.

In another broad aspect of the present technology, there is provided a method for near real-time image enhancement of endoscopic images, the method executable by at least one processor, the method comprising: accessing a first image from a stream of images; determining that a contaminated segment is present within the first image; determining at least one of (i) a type of contaminant associated with the contaminated segment or (ii) a dimension of the contaminated segment; adjusting a resolution of the first image, the adjusting being based on the at least one of (v) the type of contaminant or (vi) the dimension of the contaminated segment, the adjusting resulting in generating a reduced-resolution image from the first image; inputting, to a machine-learning algorithm (MLA), (ix) the reduced-resolution image to generate a replacement segment for the contaminated segment; and generating a restored image based on the replacement segment.

In some embodiments of the method, the method further comprises: accessing a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image ; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the method, the method further comprises displaying the restored image.

In another broad aspect of the present technology there is provided a method for near real-time image enhancement of endoscopic images, the method executable by at least one processor, the method comprising: accessing a first image from a stream of images; determining that a contaminated segment is present within the first image; determining at least one of (i) a type of contaminant associated with the contaminated segment or (ii) a dimension of the contaminated segment; adjusting a frame rate associated with the stream of images, the adjusting being based on the at least one of (v) the type of contaminant or (vi) the dimension of the contaminated segment, the adjusting resulting in a reduced stream of images; inputting, to a machine-learning algorithm (MLA), at least one extracted image taken from the reduced stream of images to generate a replacement segment for the contaminated segment; and generating a restored image based on the replacement segment.

In some embodiments of the method, the method further comprises: accessing a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image ; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the method, the method further comprises displaying the restored image.

In another broad aspect of the present technology there is provided a method for near real-time image enhancement of endoscopic images, the method executable by at least one processor, the method comprising: accessing a first image from a stream of images; determining that a contaminated segment is present within a region of interest (ROI) of the first image; determining at least one of (i) a type of contaminant associated with the contaminated segment or (ii) a dimension of the contaminated segment; in response to the type of contaminant or the dimension of the contaminated segment being below a first threshold, estimating a time duration required for performing an image processing operation on the contaminated segment; selectively reducing, based on the estimated time duration, at least one of a resolution of the first image, a rate associated with the stream of images, and a dimension of the ROI; accessing a second image from a cache memory storing cached images; generating, based on the second image, a prediction for a replacement segment to replace the contaminated segment; composing the replacement segment onto the contaminated segment, thereby generating a composed image; selectively outputting, to a display, at least one of the first image and the composed image.

In another broad aspect of the present technology there is provided a method for managing a cache memory storing a plurality of cached images, the method comprising: accessing a first image from a stream of images; accessing the cache memory storing a plurality of cached images; determining at least one of: a temporal relevance score of the plurality of cached images, the temporal relevance score being indicative of a time elapsed between capturing an individual cached image and the first image; or a contextual relevance score of the plurality of cached images, the contextual relevance score being indicative of a degree of structural similarity between an individual cached image and the first image ; and selecting, from the plurality of cached images, a second image based on the temporal relevance score meeting a temporal relevance threshold or the contextual relevance score meeting a contextual relevance threshold; and returning the second image.

In some embodiments of the method, the method further comprises: determining whether the first image is adequate for caching, the adequacy being determined based on at least one image quality metric; and in response to determining that the first image is adequate for caching, storing the first image in the cache memory as one of the plurality of cached images.

In some embodiments of the method, the at least one image quality metric comprises one or more of: a sharpness metric, determined based on at least one of an edge gradient value and a variance of the Laplacian; or a haze level metric, determined based on at least one of a Dark Channel Prior (DCP) value and a Fog Aware Density Evaluation (FADE) value; or an information richness metric, determined based on entropy analysis.

In some embodiments of the method, the individual cached image is removed from the cache memory in response to at least one of: the temporal relevance score of the individual cached image failing to exceed the temporal relevance threshold; and the contextual relevance score of the individual cached image failing to exceed the contextual relevance threshold.

In some embodiments of the method, the method further comprises selectively inputting, the second image to a machine-learning algorithm (MLA).

In another broad aspect of the present technology, there is provided a system for near real-time image enhancement of endoscopic images, the system comprising at least one processor configured to: access a first image from a stream of images; determine that a contaminated segment is present within the first image; determine at least one of (i) a type of contaminant associated with the contaminated segment or (ii) a dimension of the contaminated segment; adjust at least one of (iii) a resolution of images from the stream of images or (iv) a frame rate associated with the stream of images, the adjusting being based on the at least one of (v) the type of contaminant or (vi) the dimension of the contaminated segment, the adjusting resulting in at least one of (vii) generating reduced-resolution images from the stream of images or (viii) a reduced stream of images; input, to a first machine-learning algorithm (MLA), (ix) at least one reduced-resolution image from the reduced-resolution images or (x) at least one extracted image taken from the reduced stream of images to generate a replacement segment for the contaminated segment; and generate a restored image based on the replacement segment.

In some embodiments of the system, the contamination type is indicative of at least one of smoke, fog, blood, fat, blur, or presence of a surgical tool.

In some embodiments of the system, the at least one processor is further configured to: access a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the system, the at least one processor is further configured to: determine whether the first image is adequate for caching, the adequacy being determined based on at least one image quality metric; and in response to determining that the first image is adequate for caching, store the first image in the cache memory as one of the plurality of cached images.

In some embodiments of the system, the at least one image quality metric comprises one or more of: a sharpness metric, determined based on at least one of an edge gradient value and a variance of the Laplacian; or a haze level metric, determined based on at least one of a Dark Channel Prior (DCP) value, or a Fog Aware Density Evaluation (FADE) value; and an information richness metric, determined based on entropy analysis.

In some embodiments of the system, an individual cached image is removed from the cache memory in response to at least one of: the temporal relevance score of the individual cached image failing to exceed the temporal relevance threshold; and the contextual relevance score of the individual cached image failing to exceed the contextual relevance threshold.

In some embodiments of the system, the at least one processor is further configured to selectively upscale the restored image.

In some embodiments of the system, the at least one processor is further configured to selectively increase the frame rate associated with the stream of images.

In some embodiments of the system, the at least one processor is further configured to selectively adjust the dimension of the region of interest (ROI) used to determine that the contaminated segment is present within the first image.

In some embodiments of the system, the at least one processor is further configured to: generate a visual cue on the restored image, the visual cue indicating at least one of: a region corresponding to the replacement segment; and a reliability score for the replacement segment within the composed image frame.

In some embodiments of the system, determining that the contaminated segment is present within the first image comprises executing a second machine-learning algorithm (MLA) by inputting the first image to the second MLA and outputting, by the second MLA, at least one of (i) a type of contaminant associated with the contaminated segment or (ii) a dimension of the contaminated segment.

In some embodiments of the system, wherein prior to determining that the contaminated segment is present within the first image, the system executes grouping the first image with another image from the stream of images.

In some embodiments of the system, determining that the contaminated segment is present is executed on some images but not all images of the stream of images.

In some embodiments of the system, adjusting at least one of (iii) the resolution of images from the stream of images or (iv) the frame rate associated with the stream of images is based on available computing resources of the system.

In some embodiments of the system, prior to inputting to the first MLA, (ix) the at least one reduced-resolution image from the reduced-resolution images or (x) the at least one extracted image taken from the reduced stream of images, the system executes: selecting the first MLA from a plurality of MLAs based on at least one of: the type of contaminant associated with the contaminated segment; or available computing resources of the system.

In some embodiments of the system, the at least one extracted image taken from the reduced stream of images is further processed to reduce its resolution before being used to generate the replacement segment for the contaminated segment.

In some embodiments of the system, the system is configured to display the restored image.

In another broad aspect of the present technology there is provided a system for near real-time image enhancement of endoscopic images, the system executable by at least one processor, the system comprising: accessing a first image from a stream of images; determining that a contaminated segment is present within the first image; determining at least one of (i) a type of contaminant associated with the contaminated segment or (ii) a dimension of the contaminated segment; adjusting at least one of (iii) a resolution of the first image or (iv) a frame rate associated with the stream of images, the adjusting being based on the at least one of (v) the type of contaminant or (vi) the dimension of the contaminated segment, the adjusting resulting in at least one of (vii) generating a reduced-resolution image from the first image or (viii) a reduced stream of images; inputting, to a machine-learning algorithm (MLA), (ix) the reduced-resolution image or (x) at least one extracted image taken from the stream of reduced images to generate a replacement segment for the contaminated segment; and generating a restored image based on the replacement segment.

In some embodiments of the system, the at least one processor is further configured to: access a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the system, the system is configured to display the restored image.

In another broad aspect of the present technology, there is provided a system for near real-time image enhancement of endoscopic images, the system comprising at least one processor configured to: access a first image from a stream of images; determine that a contaminated segment is present within the first image; determine at least one of (i) a type of contaminant associated with the contaminated segment or (ii) a dimension of the contaminated segment; adjust a resolution of the first image, the adjusting being based on the at least one of (v) the type of contaminant or (vi) the dimension of the contaminated segment, the adjusting resulting in generating a reduced-resolution image from the first image; input, to a machine-learning algorithm (MLA), (ix) the reduced-resolution image to generate a replacement segment for the contaminated segment; and generate a restored image based on the replacement segment.

In some embodiments of the system, wherein the at least one processor is further configured to: access a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the system, the system is configured to display the restored image.

In another broad aspect of the present technology there is provided a system for near real-time image enhancement of endoscopic images, the system comprising at least one processor configured to: access a first image from a stream of images; determine that a contaminated segment is present within the first image; determine at least one of (i) a type of contaminant associated with the contaminated segment or (ii) a dimension of the contaminated segment; adjust a frame rate associated with the stream of images, the adjusting being based on the at least one of (v) the type of contaminant or (vi) the dimension of the contaminated segment, the adjusting resulting in a reduced stream of images; input, to a machine-learning algorithm (MLA), at least one extracted image taken from the reduced stream of images to generate a replacement segment for the contaminated segment; and generate a restored image based on the replacement segment.

In some embodiments of the system, the at least one processor is further configured to: access a second image from a cache memory storing cached images, the accessing the second image from the cache memory comprising: determining at least one of: a temporal relevance score of the plurality of cached images, the temporal relevance score being indicative of a time elapsed between capturing an individual cached image and the first image; or a contextual relevance score of the plurality of cached images, the contextual relevance score being indicative of a degree of structural similarity between an individual cached image and the first image; and selecting, from the plurality of cached images, the second image based on the temporal relevance score meeting a temporal relevance threshold or the contextual relevance score meeting a contextual relevance threshold.

In some embodiments of the system, the system is configured to display the restored image.

In another broad aspect of the present technology, there is provided a system for near real-time image enhancement of endoscopic images, the system comprising at least one processor configured to: access a first image from a stream of images; determine that a contaminated segment is present within a region of interest (ROI) of the first image; determine at least one of (i) a type of contaminant associated with the contaminated segment or (ii) a dimension of the contaminated segment; in response to the type of contaminant or the dimension of the contaminated segment being below a first threshold, estimate a time duration required for performing an image processing operation on the contaminated segment; selectively reduce, based on the estimated time duration, at least one of a resolution of the first image, a rate associated with the stream of images, and a dimension of the ROI; access a second image from a cache memory storing cached images; generate, based on the second image, a prediction for a replacement segment to replace the contaminated segment; compose the replacement segment onto the contaminated segment, thereby generating a composed image; selectively output, to a display, at least one of the first image and the composed image.

In another broad aspect of the present technology, there is provided a system for managing a cache memory storing a plurality of cached images, the system comprising at least one processor configured to: access a first image from a stream of images; access the cache memory storing a plurality of cached images; determine at least one of: a temporal relevance score of the plurality of cached images, the temporal relevance score being indicative of a time elapsed between capturing an individual cached image and the first image; or a contextual relevance score of the plurality of cached images, the contextual relevance score being indicative of a degree of structural similarity between an individual cached image and the first image; and select, from the plurality of cached images, a second image based on the temporal relevance score meeting a temporal relevance threshold or the contextual relevance score meeting a contextual relevance threshold; and return the second image.

In some embodiments of the system, the at least one processor is further configured to: determine whether the first image is adequate for caching, the adequacy being determined based on at least one image quality metric; and in response to determining that the first image is adequate for caching, store the first image in the cache memory as one of the plurality of cached images.

In some embodiments of the system, the at least one image quality metric comprises one or more of: a sharpness metric, determined based on at least one of an edge gradient value and a variance of the Laplacian; or a haze level metric, determined based on at least one of a Dark Channel Prior (DCP) value and a Fog Aware Density Evaluation (FADE) value; or an information richness metric, determined based on entropy analysis.

In some embodiments of the system, the at least one processor is further configured to remove individual cached image from the cache memory in response to at least one of: the temporal relevance score of the individual cached image failing to exceed the temporal relevance threshold; and the contextual relevance score of the individual cached image failing to exceed the contextual relevance threshold.

In some embodiments of the system, the at least one processor is further configured to selectively input, the second image to a machine-learning algorithm (MLA).

In another broad aspect of the present technology there is provided a non-transitory computer-readable medium comprising instructions, which upon being executed by at least one processor, configure the at least one processor to execute the various methods described in this specification.

According to another aspect of the invention, there is disclosed a method of improving image quality through an endoscope, the method comprising: receiving image data comprising image frames; caching a first one of the image frames, thereby generating a cached image frame; capturing a second one of the image frames, resulting in a captured image frame; determining that the captured image frame is contaminated; identifying a contaminated image segment from the captured image frame that is contaminated; generating a prediction for a replacement image segment to replace the contaminated image segment; and composing the replacement image segment onto the cached image frame, thereby generating a composed image.

In at least one embodiment, the method further comprises replacing the segmented image frame that is contaminated with the composed image.

In at least one embodiment, the method further comprises classifying the cached image frame as a last clear frame using a first AI model for segmentation.

In at least one embodiment, the determining that the captured image frame is contaminated is based at least in part by transferring the captured image frame to a first AI model for segmentation.

In at least one embodiment, the method further comprises segmenting the contaminated image segment from the captured image frame that is obstructed.

In at least one embodiment, the method further comprises inputting the contaminated image segment into a second AI model for generation to preprocess the contaminated image segment for restoration.

In at least one embodiment, the generating the prediction for the replacement image segment to replace the contaminated image segment is based at least in part by using a second AI model for generation.

In at least one embodiment, the composing the replacement image segment onto the cached image frame is based at least in part on using a second AI model for generation to generate the composed image using the last clear frame.

According to another aspect of the invention, there is disclosed a method of improving image quality through an endoscope, the method comprising: receiving image data comprising image frames; capturing a first one of the image frames, resulting in a captured image frame; determining that the captured image frame is contaminated; segmenting a part of the captured image frame that is contaminated; generating a restored image frame based at least in part on a last clear frame and the captured image frame.

In at least one embodiment, the method further comprises replacing the captured frame that is contaminated with the restored image frame.

In at least one embodiment, the determining that the captured frame is contaminated is based at least in part on a classification by a first AI model for segmentation classifying that the captured frame is contaminated.

In at least one embodiment, the segmenting the part of the captured frame that is contaminated is done using the first AI model for segmentation.

In at least one embodiment, the method further comprises inputting the contaminated image segment into a second AI model for generation to preprocess the contaminated image segment for restoration.

In at least one embodiment, the method further comprises generating a prediction for a replacement image segment to replace the contaminated image segment for use in generating the restored image frame.

According to another aspect of the invention, there is disclosed a method of improving image quality through an endoscope, the method comprising: receiving image data comprising image frames; capturing a first one of the image frames, resulting in a first captured image frame; determining that the first captured frame is not contaminated; storing the first captured image frame as a last clear frame; capturing a second one of the image frames, resulting in a second captured image frame; determining that the second captured image frame is contaminated; and generating a restored image frame based at least in part on the last clear frame and the second captured image frame.

In at least one embodiment, the method further comprises replacing the second captured frame that is contaminated with the restored image frame.

In at least one embodiment, the determining that the first captured frame is not contaminated is done using a first AI model for segmentation.

In at least one embodiment, the method further comprises segmenting a part of the second captured image frame that is contaminated.

In at least one embodiment, the method further comprises inputting the contaminated image segment into a second AI model for generation to preprocess the contaminated image segment for restoration.

In at least one embodiment, the generating the restored image frame is also based at least in part on a second AI model for generation processing the last clear frame.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein. In the drawings:

FIG. 1 shows a block diagram of an example embodiment of a system for improving image quality through an endoscope;

FIG. 2A shows a schematic diagram of an example embodiment of a first operating environment for a system for improving image quality through an endoscope; FIG. 2B shows a schematic diagram of an example embodiment of a second operating environment for a system for improving image quality through an endoscope;

FIG. 3 shows an example embodiment of an operating room setup for minimally invasive surgery;

FIG. 4 shows a flow diagram of an example embodiment of system integration in an operating room (OR) setup;

FIG. 5 shows a block diagram of data flow for an example embodiment showing signal processing in a system for improving image quality through an endoscope;

FIG. 6 shows a block diagram of data flow between various parts of an example embodiment of a system for image improvement through an endoscope;

FIG. 7A shows a flow diagram of an example embodiment of a decision process of the system for image improvement through an endoscope;

FIG. 7B shows an example of a captured frame during the decision process of FIG. 7A; FIG. 7C shows an example of AI model #1 segmentation during the decision process of FIG. 7A; FIG. 7D shows an example of determining whether a video feed is contaminated during the decision process of FIG. 7A; FIG. 7E shows an example of AI model #2 generation during the decision process of FIG. 7A; FIG. 7F shows an example of an initial frame with a prediction output composed thereon during the decision process of FIG. 7A; FIG. 7G shows an example of a forward frame during the decision process of FIG. 7A;

FIG. 8 shows a flow diagram of an example embodiment of an image restoration process of the system for image improvement through an endoscope;

FIG. 9A shows a flow diagram of an example embodiment of a decision process of the system for image improvement through an endoscope having cache memory;

FIG. 9B shows an example of a captured frame during the decision process of FIG. 9A; FIG. 9C shows an example of AI model #1 segmentation during the decision process of FIG. 9A; FIG. 9D shows an example of determining whether a video feed is contaminated during the decision process of FIG. 9A; FIG. 9E shows an example of AI model #2 generation during the decision process of FIG. 9A; FIG. 9F shows an example of an initial frame with a prediction output composed thereon during the decision process of FIG. 9A; FIG. 9G shows an example of a last clear frame stored in memory during the decision process of FIG. 9A; FIG. 9H shows an example of a forward frame during the decision process of FIG. 9A;

FIG. 10 shows a flow diagram of an example embodiment of a method for improving image quality;

FIG. 11A shows a first example of an actual surgical view before image quality improvement; FIG. 11B shows a first example of an improved view after image quality improvement;

FIG. 12A shows a second example of an actual surgical view before image quality improvement; FIG. 12B shows a second example of an improved view after image quality improvement;

FIG. 13 shows an example of an architecture for image segmentation and restoration; and

FIG. 14 illustrates a flow diagram of an exemplary embodiment of an image enhancement process 2000 for improving visual clarity through an endoscope.

Further aspects and features of the example embodiments described herein will appear from the following description taken together with the accompanying drawings.

### DESCRIPTION OF VARIOUS EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems, or methods having all of the features of any one of the devices, systems, or methods described below or to features common to multiple or all of the devices, systems, or methods described herein. It is possible that there may be a device, system, or method described herein that is not an embodiment of any claimed subject matter. Any subject matter that is described herein that is not claimed in this document may be the subject matter of another protective instrument, for example, a continuing patent application, and the applicants, inventors, or owners do not intend to abandon, disclaim, or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical signal, electrical connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should also be noted that, as used herein, the wording “at least one of X or Y” and “at least one of X and Y” is intended to mean X or Y or both X and Y.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5%, or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

It should also be noted that the use of the term “window” in conjunction with describing the operation of any system or method described herein is meant to be understood as describing a user interface for performing initialization, configuration, or other user operations. A window may, for example, display a picture, or even a picture-in-picture, derived from an imaging device, such as a camera or medical imaging device.

The example embodiments of the devices, systems, or methods described in accordance with the teachings herein may be implemented as a combination of hardware and software. For example, the embodiments described herein may be implemented, at least in part, by using one or more computer programs, executing on one or more programmable devices comprising at least one processing element and at least one storage element (i.e., at least one volatile memory element and at least one non-volatile memory element). The hardware may comprise input devices including at least one of a touch screen, a keyboard, a mouse, buttons, keys, sliders, and the like, as well as one or more of a display, a printer, and the like depending on the implementation of the hardware.

It should also be noted that there may be some elements that are used to implement at least part of the embodiments described herein that may be implemented via software that is written in a high-level procedural language such as object-oriented programming. The program code may be written in C++, C #, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a Read Only Memory (ROM), a magnetic disk, an optical disc, a Universal Serial Bus (USB) key, and the like that is readable by a device having at least one processor, an operating system, and the associated hardware and software that is necessary to implement the functionality of at least one of the embodiments described herein. The software program code, when read by the device, configures the device to operate in a new, specific, and predefined manner (e.g., as a specific-purpose computer) in order to perform at least one of the methods described herein.

At least some of the programs associated with the devices, systems, and methods of the embodiments described herein may be capable of being distributed in a computer program product comprising a computer readable medium that bears computer usable instructions, such as program code, for one or more processing units. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

In at least some of the embodiments described herein, artificial intelligence (AI) is used to restore the endoscopic video signal in real time while integrating with equipment already available in a hospital.

At least some of the embodiments described herein have an advantage over conventional approaches because they improve the quality of minimally invasive surgery by providing an AI-based approach to address the challenge of contaminated camera lenses. To that end, AI-driven software can enhance the visual conditions in the presence of camera lens contamination, ensuring that surgeons have improved visibility during procedures.

AI-driven software is advantageous as it addresses an unmet need for clear vision in minimally invasive surgery. The lack of automation in conventional cleaning processes brings frustration, increased errors, and poorer patient outcomes. AI-driven software can improve the cleaning process by reducing the number of cleaning events through image enhancement.

Using AI-based image processing, images may be actively enhanced in real time. Cleaning events may be delayed or eliminated, thereby minimizing the number of cleaning events and time lost from cleaning.

The image enhancement can allow for the reduction of disruptions in surgery.

At least some of the embodiments described herein have benefit that they monitor and improve the current image quality through the endoscope. On one hand, the monitoring of the image quality may enable surgeons to make better decisions as to when to clean the endoscope. On the other hand, the improvement of the image quality may reduce the frequency needed to clean the endoscope while providing adequate image quality throughout the surgery.

At least some of the embodiments described herein automatically enhance the visualization of any camera display in real time during surgery in order to reduce the impairment caused by contaminants to reduce the number of cleaning events during a procedure. At least some of these embodiments are systems or methods residing on an edge platform connected between the endoscopic processor and the display. These systems or methods determine when a cleaning event is necessary, for example, by providing an API to integrate with third-party cleaning systems. These systems or methods also use computer vision algorithms to reconstruct in real time the image of a contaminated camera view.

At least some of the embodiments described herein provide technical benefits, such as being non-disruptive, versatile, or universal.

These embodiments are non-disruptive because processing is done automatically and in real time so that no intervention is required from the surgical team.

These embodiments are versatile because any type of lens contamination is addressed, such as fog, smoke, blood, fluids, etc.

These embodiments are universal because the systems and methods run on any endoscopic process. Alternatively, or in addition, the system and methods are platform-agnostic, such that they may be used with any comparable medical imaging platform.

In at least some of the embodiments described herein, the systems or methods do not process a fully obstructed camera view; however, they do not aim to do so. In such a case, the goal is to reduce the number of necessary cleaning events by enhancing the image quality in real time.

In at least some of the embodiments described herein, the systems or methods provide a systematic approach to improving visual conditions in surgery. They provide a technical approach to addressing all types of contamination. They may mitigate erratic reconstructions, also referred to as “hallucinations” (where a model output would not exist in real life), for example, by restoring the contaminated part of the image while leaving the rest of the image unchanged.

In accordance with the teachings herein, there are provided various embodiments for a system for improving image quality through an endoscope, as well as the methods, and computer products for use therewith.

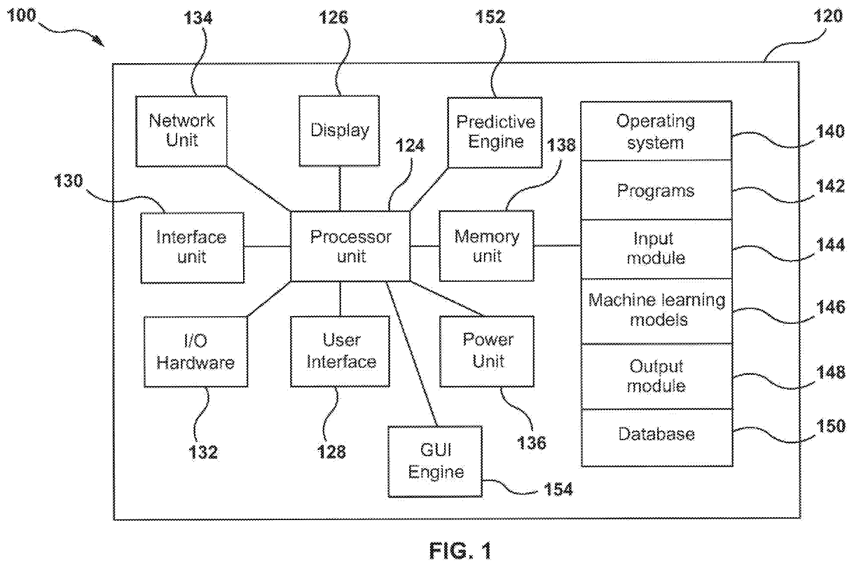

Reference is first made to FIG. 1, showing a block diagram of an example embodiment of system 100 for improving image quality through an endoscope. The system 100 includes at least one server 120. The server 120 may communicate with one or more user devices (not shown), for example, wirelessly or over the Internet. The system 100 may also be referred to as a machine learning system when used as such.

The user device may be a computing device that is operated by a user. The user device may be, for example, a smartphone, a smartwatch, a tablet computer, a laptop, a virtual reality (VR) device, or an augmented reality (AR) device. The user device may also be, for example, a combination of computing devices that operate together, such as a smartphone and a sensor. The user device may also be, for example, a device that is otherwise operated by a user, such as a drone, a robot, or remote-controlled device; in such a case, the user device may be operated, for example, by a user through a personal computing device (such as a smartphone). The user device may be configured to run an application (e.g., a mobile app) that communicates with other parts of the system 100, such as the server 120.

The server 120 may run on a single computer, including at least one processor unit 124, a display 126, a user interface 128, an interface unit 130, input/output (I/O) hardware 132, a network unit 134, a power unit 136, and a memory unit (also referred to as “data store”) 138. In other embodiments, the server 120 may have more or less components but generally function in a similar manner. For example, the server 120 may be implemented using more than one computing device.

The at least one processor unit 124 may include a standard processor, such as the Intel Xeon processor, for example. Alternatively, there may be a plurality of processors that are used by the at least one processor unit 124, and these processors may function in parallel and perform certain functions. The display 126 may be, but not limited to, a computer monitor or a Liquid Crystal Display (LCD) display such as that for a tablet device. The user interface 128 may be an Application Programming Interface (API) or a web-based application that is accessible via the network unit 134. The network unit 134 may be a standard network adapter such as an Ethernet or 802.11x adapter.

The at least one processor unit 124 may execute a predictive engine 152 that functions to provide predictions by using machine learning models 146 stored in the memory unit 138. The predictive engine 152 may build a predictive algorithm through machine learning. The training data may include, for example, image data, video data, audio data, and text.

The at least one processor unit 124 can also execute a graphical user interface (GUI) engine 154 that is used to generate various GUIs. The GUI engine 154 provides data according to a certain layout for each user interface and also receives data input or control inputs from a user. The GUI then uses the inputs from the user to change the data that is shown on the current user interface or changes the operation of the server 120 which may include showing a different user interface.

The memory unit 138 may store the program instructions for an operating system 140, program code 142 for other applications, an input module 144, a plurality of machine learning models 146, an output module 148, and a database 150. The machine learning models 146 may include, but are not limited to, image recognition and categorization algorithms based on deep learning models and other approaches. The database 150 may be, for example, a local database, an external database, a database on the cloud, multiple databases, or a combination thereof.

In at least one embodiment, the machine learning models 146 include a combination of convolutional and recurrent neural networks. Convolutional neural networks (CNNs) may be designed to recognize images or patterns. CNNs can perform convolution operations, which, for example, can be used to classify regions of an image, and see the edges of an object recognized in the image regions. Recurrent neural networks (RNNs) can be used to recognize sequences, such as text, speech, and temporal evolution, and therefore RNNs can be applied to a sequence of data to predict what will occur next. Accordingly, a CNN may be used to read what is happening on a given image at a given time, while an RNN can be used to provide an informational message.

The programs 142 comprise program code that, when executed, configures the at least one processor unit 124 to operate in a particular manner to implement various functions and tools for the system 100.

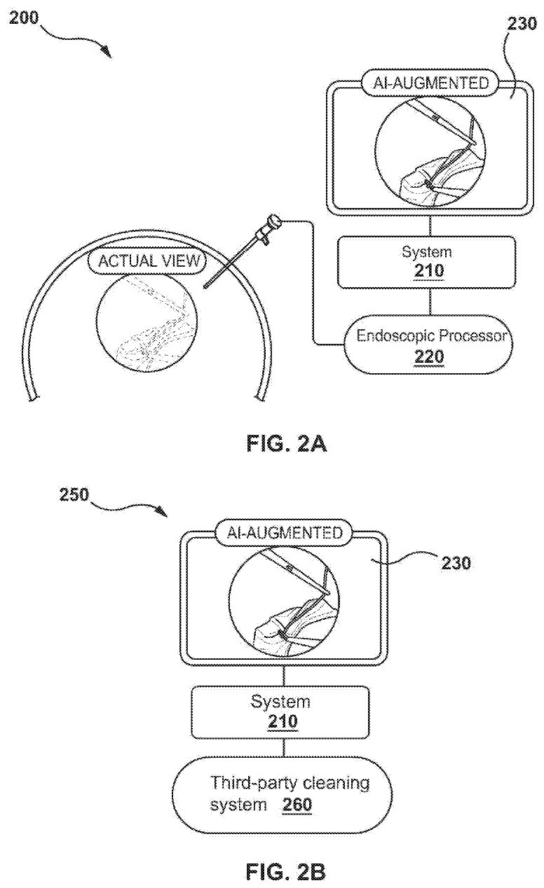

FIG. 2A shows a schematic diagram of an example embodiment of a first operating environment 200 for a system 210 for improving image quality. FIG. 2B shows a schematic diagram of an example embodiment of a second operating environment 250 for the system 210. Some or all of the system 210 may be implemented using some or all of the system 100. Some or all of the endoscopic processor 220 may be implemented using some or all of the system 100. The system 210 comprises a video processor connected between the endoscopic processor 220 and the display 230 using, for example, standard connectivity. The system 210 embeds a software module enhancing the image quality in real time. The software module may use an AI trained on synthetic data to enhance the clarity of the image and remove contaminants when the view is partially obstructed. The software module may also use an AI to determine when a cleaning event is desirable, for example, when the lens is fully obstructed. The software module may provide an API for integrating with third-party cleaning systems.

Alternatively, the system 210 may be embedded in the endoscopic processor 220. In such a case, the functionality of the system 210 is realized by the endoscopic processor 220 having embedded therein some or all of the system 100 to provide the endoscopic processor 220 the hardware and/or software required to carry out the operations, steps, and/or actions for vision improvement described herein.

In some embodiments, the system 210 may comprise Audio-Visual over Internet Protocol (AVoIP) capabilities. The system 210 may be integrated with an existing AVoIP platform. The system 210 may be configured to communicate with (or work with) an AVoIP platform. For example, the system 210 may provide vision improvement for endoscopy, digital operating room (OR) applications, and/or a catheterization lab. Also for example, the system 210 may support various types of medical procedures, which may include such technologies as 4K video routing with IP-based fiber optic video switching.

Advantageously, the system 210 may provide a real-time AI-enhanced surgical view with seamless endoscopic system integration. Advantageously, the system 210 may reduce scope removals, optionally up to 40%. The inventors saw benefits of 40% of scope removal after conducting an internal analysis of retrospective videos of sinus surgery where it was estimated, based on a visual appreciation of blurriness, that 40% of the scope removals could have been prevented using the methods for improving image quality as described herein.

The endoscopic processor 220 may receive input from a camera built into the endoscope when the endoscope is inserted into a gastrointestinal tract (or other human body part). The endoscopic processor 220 may receive image signals from the endoscope, which may be processed to be displayed or otherwise output. The endoscopic processor 220 may control the processing of image signals from the endoscope and accordingly function as an image processor.

The endoscopic processor 220 may receive input from the endoscope, such as buttons that can be pressed by the user to send input signals to control the endoscope or the camera built into the endoscope. These buttons may be programmed buttons that are actuated by the user in order to send an input signal to the endoscopic processor 220 to control the endoscopy video stream or image stream.

The endoscopic processor 220 may be a microcontroller, microprocessor, or microcomputer, and may employ a field-programmable gate array (FPGA) or application-specific integrated circuit (ASIC). The endoscopic processor 220 may be, for example, an NVIDIA™ Jetson™ microcomputer, which may include a software accelerator (e.g., Raspberry Pi™, TensorFlow™, etc.).

Referring now to FIG. 2B, the system 210 and a third-party cleaning system 260 provide AI-augmented visualization for slight impairment (e.g., to prevent 40% of scope removals). The system 210 and the third-party cleaning system 260 provide a contamination detection API for third-party integration and automate cleaning for heavy impairment, which may advantageously allow non-disruptive workflow.

The system 210 may advantageously enhance the endoscopic video signal in real time. Some of the challenges addressed by the system 210 and its functionality (e.g., with reference to the data flow and methods described herein) include: (a) processing at very low latency; and (b) building AI models fitting embedded constraints.

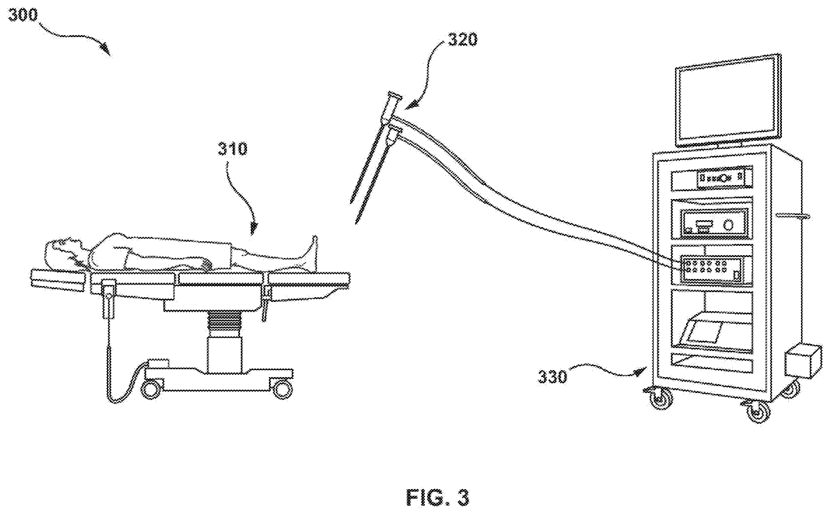

FIG. 3 illustrates an example embodiment of an operating room setup 300 for minimally invasive surgery. The operating room setup 300 comprises an operation table 310, surgical instruments 320, and an endoscopic tower 330. The endoscopic tower 330 has shelves on which to put various hardware, such as the endoscopic processor 220, the monitor 230, and additional consoles if required.

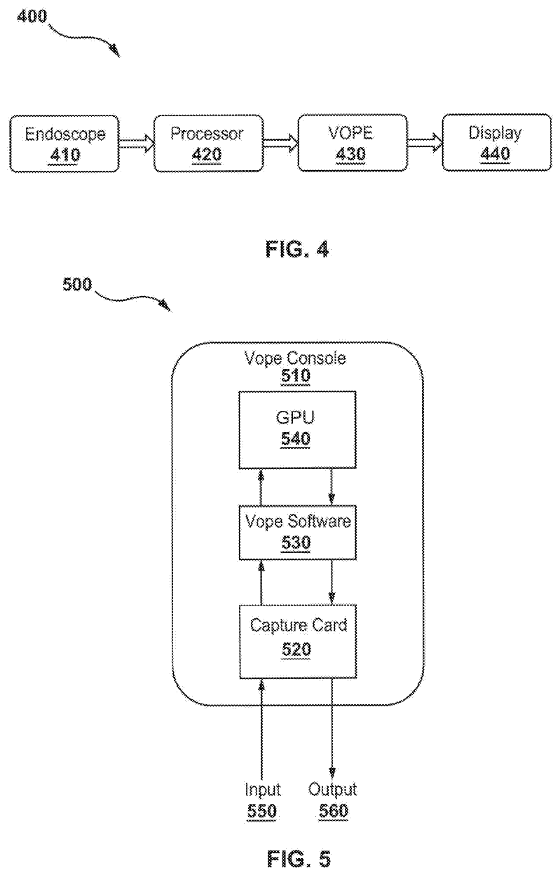

Referring now to FIG. 4, an endoscope 410, an endoscopic processor 420, and a display 440 are connected through common video interface standards such as Digital Visual Interface (DVI), Serial Digital Interface (SDI) or (High-Definition Multimedia Interface) HDMI. The supported video formats may range, for example, from 1080i or 1080p to 4K resolution. The VOPE 430, also referred to as the system 430, is connected between the endoscopic processor 420 and the display 440, processing the signal in real time to restore the video feed when there is contamination. Some or all of the system 430 may be implemented using some or all of the system 210 and/or some or all of the system 100. The system 430 may be considered to be “middleware” or a “bridge”, as found in the video industry.

The endoscope 410 may be an endoscope that is suitable for insertion into the body of a patient. In alternative embodiments, the endoscope 410 may be replaced with another imaging device and/or sensors for other medical applications and/or imaging modalities. These imaging modalities may be, for example, arthroscopy (e.g., for orthopedics), bronchoscopy (e.g., for respirology), colposcopy (e.g., for obstetrics & gynecology), crystography (e.g., for urology), CT scan (e.g., for cardiology, ENT, obstetrics & gynecology, respirology), cystoscopy (e.g., for urology), laparoscopy (e.g., for general surgery, obstetrics & gynecology), laryngoscopy (e.g., for ENT), MRI (e.g., for ENT, obstetrics & gynecology, respirology), X-ray (e.g., for obstetrics & gynecology, respirology).

The endoscopic processor 420 may be the same as the endoscopic processor 220 described herein. In alternative embodiments, when the endoscope 410 is replaced with another imaging device and/or sensors for other medical applications and/or imaging modalities, the endoscopic processor 420 may be replaced by a medical imaging processor suitable for the corresponding imaging modality. In such a case, the endoscopic processor 420 may be referred to more generally as a medical imaging processor 420.

The system 430 may be a medical grade console comprising a capture card configured to acquire and forward a video signal and a powerful Graphics Processing Unit (GPU) to run the AI models.

The system 430 may operate as an off-the-shelf AI platform. For example, the system 430 may serve as an AI solution that comprises pre-built software applications designed for the specific task of image quality improvement on its own or integrated into other general or broad range tasks. As an off-the-shelf AI platform, it may provide the benefit of rapid deployment. As an example, the system 430 may be implemented using a mini tower or standalone medical computer such as USM-500. Some of the benefits may include that it is autonomous in development, OR read, and/or IEC 60601 compliant. Integration of the system 430 into an off-the-shelf platform such as the Advantech USM-500 may be facilitated, for example, by a software development kit (SDK) such as NVIDIA Holoscan Clara. Such SDKs are designed to facilitate the integration of AI applications in real time on top of sources such as an endoscopic video signal.

The system 430 may operate as an Original Equipment Manufacturer (OEM) AI platform. For example, the system 430 may serve as an AI solution that comprises software applications that can be implemented by hardware and/or software manufacturers for the purpose of bundling with existing offerings. As an example, the system 430 may be implemented using an AI-based endoscopy module such as a Medtronic GIGenius. One of the benefits may include that it is scalable without requiring additional technical customization.

FIG. 5 illustrates a block diagram of data flow 500 for an example embodiment showing signal processing in the system for image improvement. The data flow 500 shown is between a console 510 and an input signal 550 and an output signal 560. The console 510 comprises a capture card 520 that interfaces with console software 530 which in turn utilizes at least one processor 540. The input signal 550 is first acquired and decoded by the capture card 520 and sent to the console software 530, which in turn sends data representing the input signal 550 to the at least one processor 540 to be processed by the AI models. Then a prediction is sent back to the console software 530 which composes the prediction with the initial result and sends the result to the capture card 520 to be encoded as a video stream to the monitor as the output signal 560. Some or all of the data flow 500 may be accomplished using some or all of the system 430, some of all of the system 210, or some or all of the system 100.

The capture card 520 may be a video capture card that supports one or more video modes, such as HDMI, DisplayPort, Single or dual-link DVI, 3G-SDI, HD-SDI, RGB, Component YPbPr, Composite video, and/or S-Video. For example, the capture card 520 may comprise DisplayPort 1.2, SDI, and HDMI inputs with 4K capture up to 60 frames per second, 10-bit color transfer, and HDCP compliance.

The console software 530 may include image analysis algorithms, such as object detection. The object detection may be based on YOLOv4, which uses a convolutional neural network (CNN) for performing certain functions. The YOLOv4 object detection algorithm may be advantageous as it may allow the image analysis at a faster rate. The YOLOv4 object detection algorithm may be implemented, for example, by a microcomputer with a software accelerator. For example, U-Net may be used for object segmentation. Object detection may identify objects and their locations with bounding boxes, while segmentation may delineate object boundaries at the pixel level, distinguishing between different instances (instance segmentation) or assigning class labels to pixels (semantic segmentation).

The at least one processor 540 may be a GPU optimized for machine learning, such as an NVIDIA GPU (e.g., with NVIDIA CUDA cores) or on the cloud using AWS GPU. The at least one processor 540 may be combined with, or configured to operate with, a suitable CPU (e.g., NVIDIA Camel ARM). In such a case, the at least one processor 540 may operate in combination with the CPU to run one or more of the programs 142, the machine learning models 146, and the predictive engine 152.

The data flow 500 advantageously addresses the issue of latency of signal processing so it can be used in real time. A typical goal is to remain under 100 to 300 milliseconds depending on the task at the “glass-to-glass” level, which is a reference to the glass of the lens to the glass of the display. Above these thresholds, a user may start noticing a delay between their actions and what they see on the display. This reduction in latency may be achieved by building and/or using AI models fitting embedded constraints.

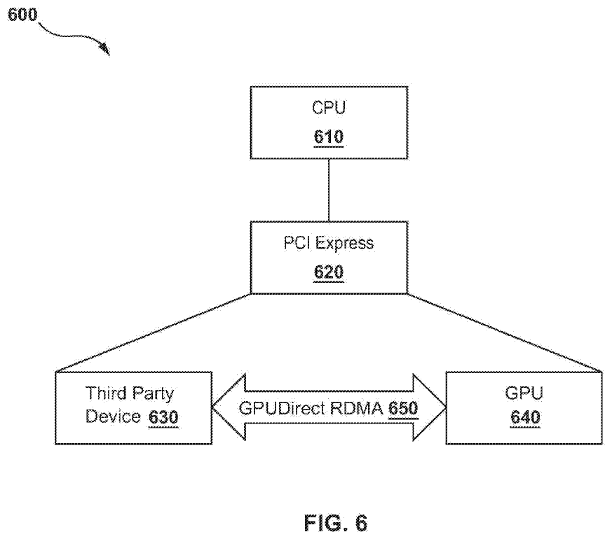

FIG. 6 shows a block diagram of data flow 600 between various parts of an example embodiment of a system for image improvement through an endoscope. The data flow 600 may be accomplished using some or all of the system 430, some or all of the system 210, and/or some or all of the system 100. The data flow 600 includes a first processor 610 (which may be, for example, a CPU) that is in communication with a (Peripheral Component Interconnect) PCI express 620. The PCI express 620 is in communication with a third-party device 630 and a second processor 640 (which may be, for example, a GPU). The third-party device 630 and the second processor 640 communicate using Remote Direct Memory Access (RDMA) 650 (e.g., GPUDirect RDMA). In order to minimize latency, the data flow 600 benefits from RDMA, which allows multiple hardware components on the same PCI bus to share the same memory space to prevent data from being transferred by the first processor 610 (also known as PCI bottleneck) as shown in FIG. 6. This way, the data remains in the same space and does not suffer from the latency introduced by moving the data to the different hardware components. A low AI model latency is also advantageous in the process to restore the contaminated image in a timely fashion. Considering that the latency of an endoscopic system is around 50 milliseconds, and also considering the I/O delays, an AI model latency around 30 milliseconds is capable to preserve a real-time experience. The data flow 600 may be considered to provide “zero-copy communication”, enabled by RDMA, where the data does not move in the hardware to minimize latency.

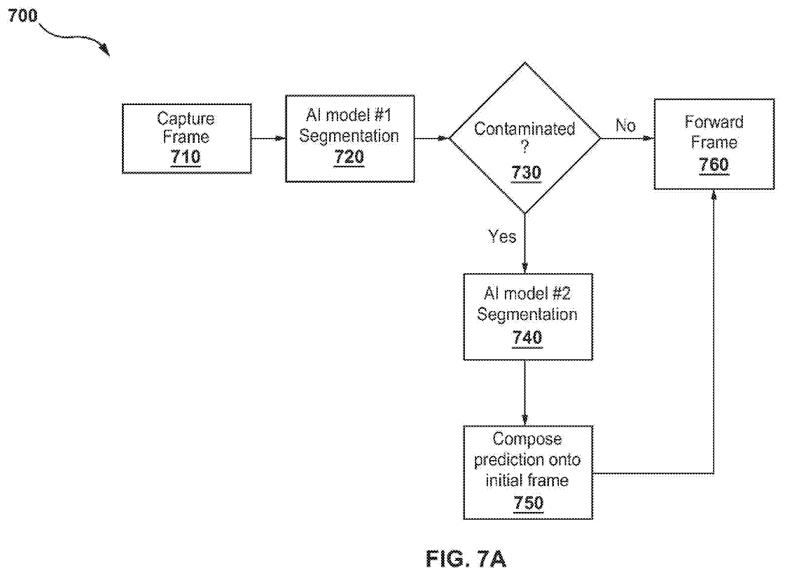

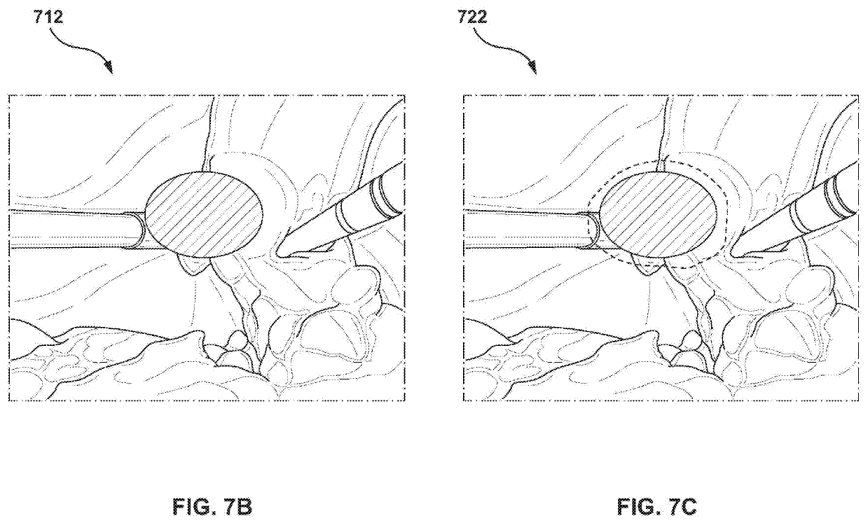

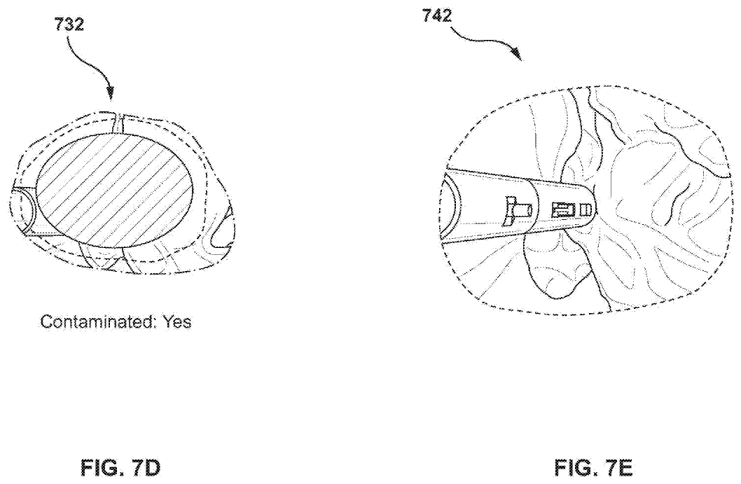

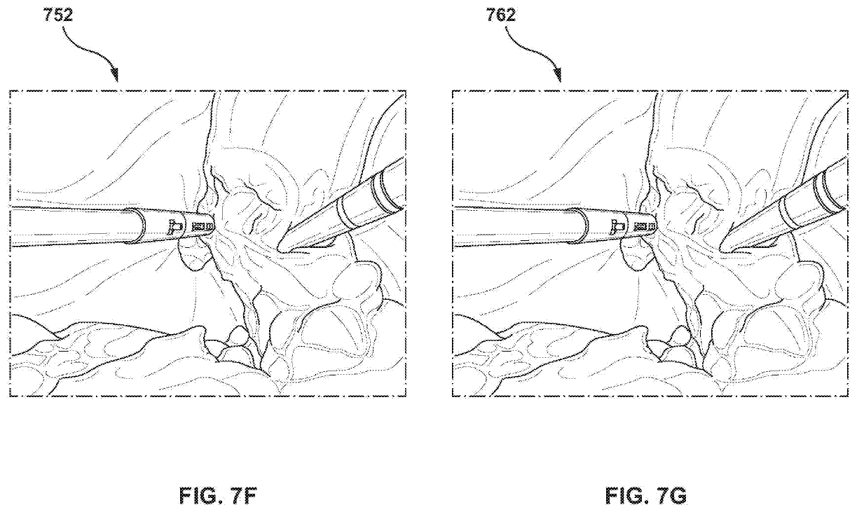

FIG. 7A illustrates a flow diagram of an example embodiment of a decision process 700 of the system for vision improvement through an endoscope. The process 700 may be accomplished using some or all of the system 430, some or all of the system 210, and/or some or all of the system 100. The process 700 corrects (or enhances) the endoscopic video signal when it is obstructed by any kind of contaminants such as blood, fog, blur, fluids, etc.

In the process 700, the system decodes the video signal (which may come, for example, from a DVI/SDI/HDMI interface) and converts it into individual frames at the same rate (e.g., at 30 FPS) to be processed by the system at capture frame 710 block (see image 712 in FIG. 7B). As an example, the surgical instruments 320 are used to visualize a minimally invasive cholecystectomy but the view is obstructed by blur (e.g., fog, condensation, sinus fluid, residue from touching internal organ structures) caused by the difference of temperature between the operating room and the patient.

The captured frame is converted to RGB 8-bit as an example and is first transferred to an AI model #1 segmentation 720 (see image 722 in FIG. 7C) that determines if the video feed is contaminated or not 730 (see image 732 in FIG. 7D) and also segments the part of the frame that is obstructed.

As an example, AI model #1 segmentation 720 is implemented using U-Net or any convolutional neural network (CNN) able to segment images and provide a classification if the image is contaminated or not. In the case of the contaminated cholecystectomy frame, the model only segments the contaminated (e.g., blurry) area of the image. If the AI model #1 segmentation 720 does not find any contamination, the frame is forwarded as is to a display monitor by a forward frame 760. If contamination is detected, the segmented area is sent to an AI model #2 generation 740 (see image 742 in FIG. 7E) responsible for restoring the contaminated part. In other words, the system may input the contaminated image segment into the AI model #2 generation 740 to preprocess the contaminated image segment for restoration.

As an example, AI model #2 generation 740 is implemented with a Generative Adversarial Network (GAN) or any model in the family of generative models (e.g., designed to restore, deblur, or denoise images). This model is trained by feeding it pairs of images, a non-contaminated image, and the same image artificially contaminated by a filter or a generative model trained to contaminate images. In the example of restoring blurred images, the model can be trained on clear images and their synthetically contaminated images equivalent using a Gaussian Blur filter. An image restoration model may comprise tens to hundreds of convolutional layers for feature extraction, followed by pooling layers for spatial down-sampling, then convolutional layers for feature refinement, possibly augmented with skip connections to preserve finer details, and finally upsampling layers to reconstruct the image to its original resolution, often integrated with techniques like residual learning or attention mechanisms for enhanced performance and fine-grained restoration.

The prediction outputted by the AI model #2 generation 740 is composed onto the initial frame 750 (see image 752 in FIG. 7F) before being forwarded to the display monitor at the forward frame 760 (see image 762 in FIG. 7G). An example of composition is by substituting the pixel values of the contaminated part identified by AI model #1 segmentation 720 of the original image with the prediction from AI model #2 generation 740. In the case of the contaminated cholecystectomy frame, the blur area(s) pixel values are substituted with the clarified ones coming from AI model #2 generation 740.

This same composition can also be achieved with alpha blending with a different level of transparency to indicate the area being corrected. Alpha blending is a technique used in computer graphics and image processing to combine two images or layers by blending their pixel values based on a specified transparency value known as the alpha channel. The alpha channel represents the degree of transparency or opacity for each pixel in an image. In the context of alpha blending, each pixel has four components: red, green, blue, and alpha (RGBA). The alpha channel determines how much of the pixel's color should contribute to the final blended result. The alpha value typically ranges from 0 (completely transparent) to 1 (completely opaque).

In other embodiments, additional or alternative markings on the restored area or somewhere else on the display can be achieved to fulfill the same purpose. The process 700 advantageously enables the image restoration mechanism only when it is necessary in order to mitigate inconsistencies such as artifacts when the vision is actually clear.

The segmentation of an image may be accomplished using an algorithm for segmenting an image into regions. One example of such an algorithm includes dividing the image into a grid of rectangular areas. For example, an image having a resolution of 640×480 may be divided into 100 regions having dimensions of 64×48 or 3072 regions having dimensions of 10×10.

Another example of a segmentation algorithm includes dividing the image into a set of segments (or contours), where each of the pixels in a region are similar with respect to some characteristic or computed property, such as color, intensity, or texture.

Alternatively, or in addition, the segmentation of an image may be accomplished using an AI trained for segmentation. For example, a training set of annotated images may be fed into a neural network that learns from the annotated images how to segment a particular image. This kind of supervised learning may then take the place of or even improve on an existing algorithm or human-based (expert, such as by a surgeon) medical image segmentation.

After an image is segmented (e.g., in accordance with any one or more of the techniques described herein), the determination of contamination within the image may be accomplished using an algorithm for determining whether the image is contaminated or whether one or more of the regions of the image is contaminated. One example of such an algorithm includes determining and assigning a blurriness score to each region and calculating a contamination score using a formula based on the blurriness scores. This formula may be, for example, a count, a sum, an average, or a weighted average of all or some of the blurriness scores. The blurriness scores may be calculated, for example, using a rule-based approach, such as a deviation from a norm for the category of segment assigned to each region. The blurriness scores may also be calculated, for example, using a variance of the Laplacian of the image. When using the Laplacian, the algorithm may address a blurriness level of 50 computed from the variance of the Laplacian over the contaminated region of the image.

Alternatively, or in addition, the determination of contamination within the image may be accomplished using an AI trained for classifying the image or regions within the image as being contaminated. For example, a training set of annotated images may be fed into a neural network that learns from the annotated images how to classify a particular image or regions within the image as being contaminated. This kind of supervised learning may then take the place of or even improve on an existing algorithm or human-based (expert, such as by a surgeon) medical image contamination determination.

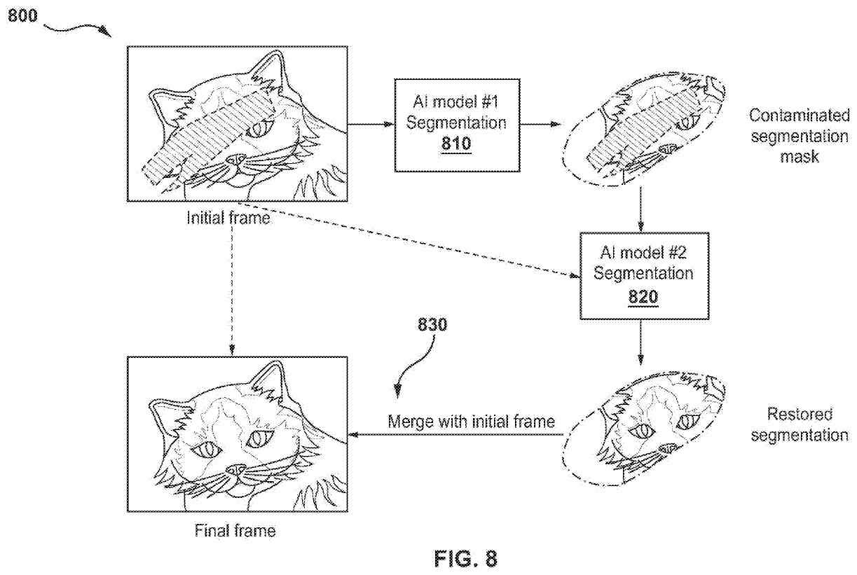

FIG. 8 illustrates a flow diagram of an example embodiment of an image restoration process 800 of the system for vision improvement through an endoscope. The process 800 may be accomplished using some or all of the system 430, some or all of the system 210, and/or some or all of the system 100. In order to mitigate inconsistencies, the process reconstructs the portion of the image that is contaminated. In the process, the initial contaminated frame is processed by an AI model #1 segmentation 810 to identify and segment the contaminated part which becomes the output of the AI model #1 segmentation 810. This segmented area is then sent to an AI model #2 generation 820 responsible for restoring the image area; to provide even more context to the model, the initial frame is an input in order to improve the accuracy of the restoration. The output for this second AI model is the restored area that is then added on top of the initial image step 830. This way, the process 800 leaves the clear part of the image untouched to prevent unnecessary restorations.

The AI model #1 segmentation 810 and/or the AI model #2 generation 820 may be trained using an image analysis training algorithm. This algorithm may employ, for example, an encoder that compresses input into feature vectors (e.g., using a CNN). The algorithm may then input the feature vectors into a decoder that reconstructs a high-resolution image from the low-resolution feature vector. The algorithm may then map the feature vectors into a distribution of target classes. The algorithm may employ supervised training, semi-supervised training, or unsupervised training.

The AI model #1 segmentation 810 and/or the AI model #2 generation 820 may use a CNN that has a convolution block, a deconvolution block, and a classifier block. The convolution block produces features, and it may consist of convolutional layers, activation layers, and pooling layers. The deconvolution block adds information to the features to allow reconstruction of images, and it may consist of convolutional layers, transposed convolution layers, and activation layers. The classifier block produces classes of objects in an image being analyzed, and it may consist of convolutional layers, activation layers, and a fully connected layer.

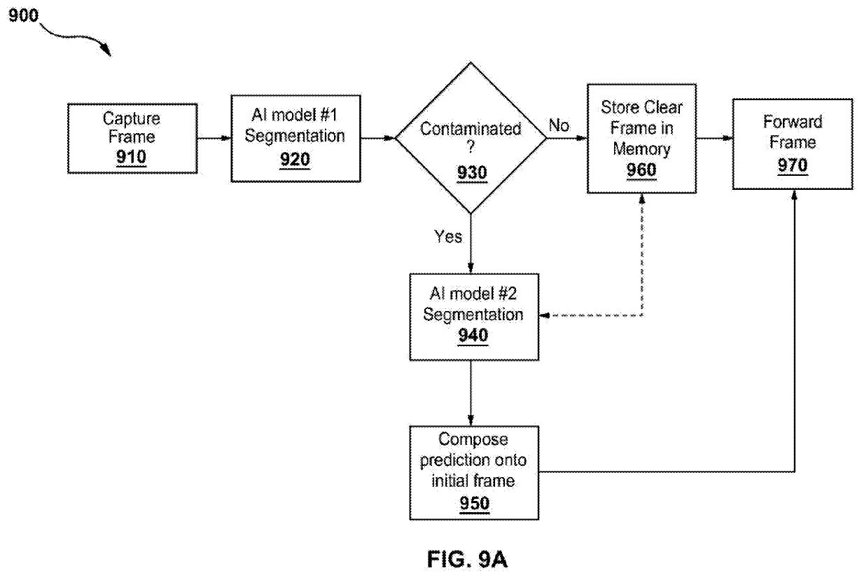

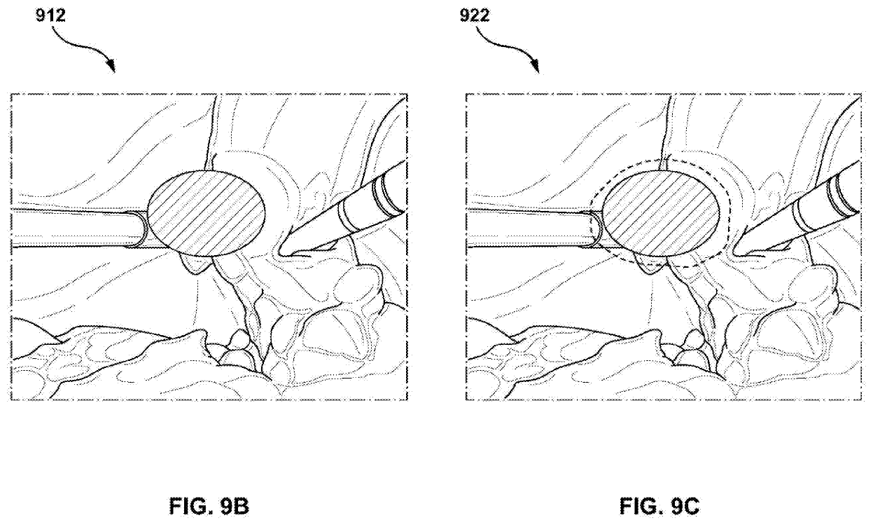

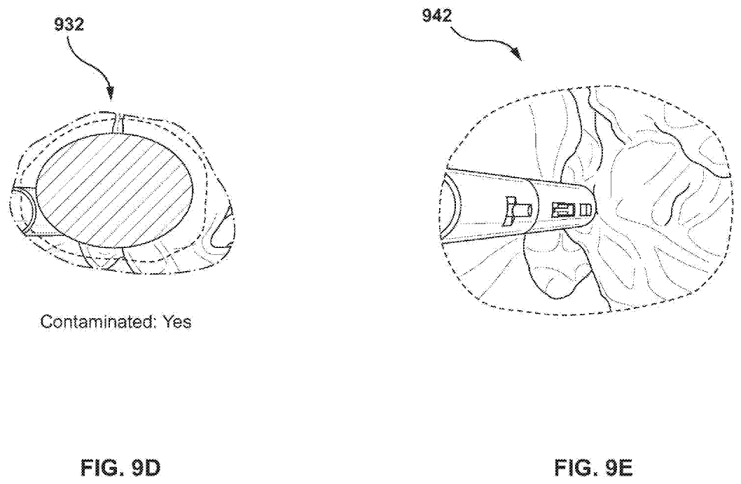

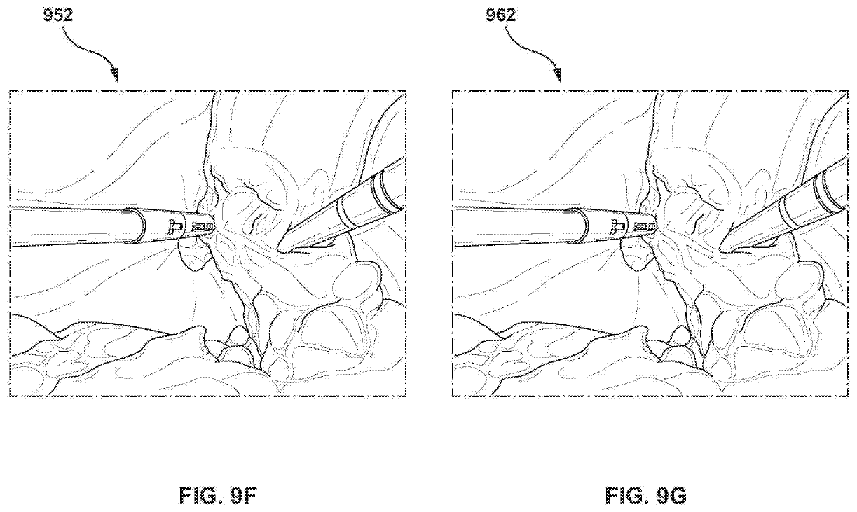

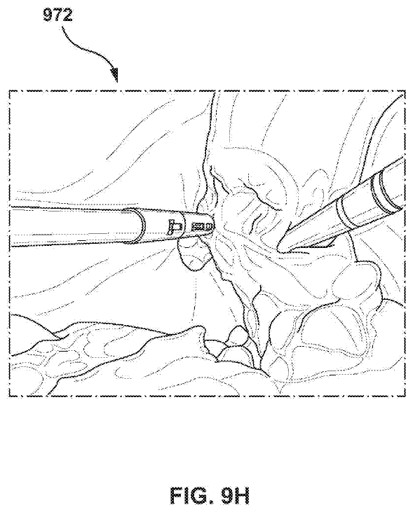

FIG. 9A illustrates a flow diagram of an example embodiment of a decision process 900 of the system for image improvement through an endoscope having cache memory. The process 900 may be accomplished using some or all of the system 430, some or all of the system 210, and/or some or all of the system 100. While the process 900 can minimize inconsistencies and increase the spatial awareness of the model, the system for improving vision may also, for example, store in memory the previous frames before the video gets contaminated in order to increase temporal awareness of the output.

The process 900 restores the endoscopic video signal when it is obstructed by any kind of contaminants such as blood, fog, blur, fluids, etc. In the process 900, the system decodes the video signal (which may come, for example, from a DVI/SDI/HDMI interface) and converts it into individual frames at the same rate (e.g., at 30 FPS) to be processed by the system at capture frame 910 block (see image 912 in FIG. 9B). The captured frame is first transferred to an AI model #1 segmentation 920 (see image 922 in FIG. 9C) that determines if the video feed is contaminated or not 930 (see image 932 in FIG. 9D) and also segments the part of the frame that is contaminated (e.g., obstructed). If the AI model #1 segmentation 920 does not find any contamination, the frame is stored in memory as a last clear frame in memory 960 (see image 962 in FIG. 9G). An example of implementation is random-memory access in any memory storage large enough to store frames. Then the frame is forwarded to a display monitor by a forward frame 970 (see image 972 in FIG. 9H). If contamination is detected, the segmented area is sent to an AI model #2 generation 940 (see image 942 in FIG. 9E) responsible for restoring the contaminated part. In other words, the system may input the contaminated image segment into the AI model #2 generation 940 to preprocess the contaminated image segment for restoration. The prediction outputted by this same model is composed onto the initial frame 950 (see image 952 in FIG. 9F) before being forwarded to the display monitor at the forward frame 970. To reuse the cholecystectomy example described with reference to process 700, when a frame is classified as clear by AI model #1 segmentation 920, it is stored in memory 960 as the “last clear frame”. When the frame gets contaminated (e.g., by blur), this “last clear frame” that will be retrieved and provided to AI model #2 generation 940 for a more faithful restoration. Memory 960 can also store multiple clear frames in a First-In-First-Out (FIFO) data structure and retrieve the best match based on Structural Similarity (SSIM) or any method computing similarities between images. After a timeout, the first frames stored in memory 960 are freed up for space.

While the process 700 captures flow of data using a simplified architecture describing how frames are processed, the process 900 adds another step by storing in memory frames that are not detected as stored clear frames in memory 960. Then, when the video feed gets obstructed, the process 900 fetches the most recent and similar frame in the cache to feed the restoration model with. This way, the model can provide a more realistic restoration based on what the system has previously seen.

In at least some embodiments of the system for improving image quality described herein, the system integrates with existing equipment in an operating room. The system also describes mitigation strategies to reduce erratic reconstructions by: (1) only restoring frames when they are detected as “contaminated”; (2) only restoring the contaminated part of a frame; and (3) caching previous clear images to enable restorations based on what the system has previously seen. These mitigation strategies are advantageous as they may improve the accuracy of the video signal and/or enhance the accuracy of restored images.

In at least one implementation of the process 900, the process 900 is carried out using the following steps: (a) receiving image data comprising image frames; (b) capturing a first one of the image frames, resulting in a first captured image frame; (c) determining that the first captured frame is not contaminated; (d) storing the first captured image frame as a last clear frame; (e) capturing a second one of the image frames, resulting in a second captured image frame; (f) determining that the second captured image frame is contaminated; and (g) generating a restored image frame based at least in part on the last clear frame and the second captured image frame. It should be appreciated that the process 900 may be carried out in a different order as described above, with fewer steps (e.g., with one of the steps omitted), or with more steps (e.g., with additional or intervening steps).

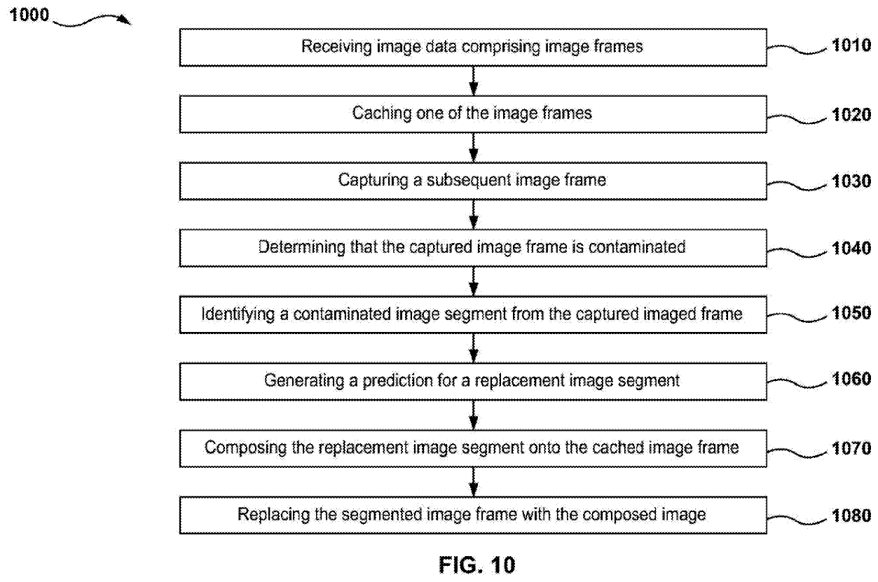

FIG. 10 illustrates a flow diagram of an example embodiment of a method 1000 for improving image quality. The method 1000 may be accomplished using some or all of the system 430, some or all of the system 210, and/or some or all of the system 100. While the method 1000 can minimize inconsistencies and increase the spatial awareness of the model, the system for improving image quality may also, for example, store in memory the previous frames before the video gets contaminated in order to increase temporal awareness of the output.

At 1010, the system receives image data comprising image frames. The image frames may be, for example, a sequence of image frames coming from a video feed. The image frames may be the result of a video signal being converted into individual frames.

At 1020, the system caches a first one of the image frames, thereby generating a cached image frame. The system may cache multiple image frames. The system may determine to cache multiple image frames, for example, on a periodic basis or based on a difference (e.g., a predetermined difference, or a difference based on anatomical changes) from a previous cached image frame. The system may use an AI model #1 (for segmentation) to classify the cached image frame as a “last clear frame”.

At 1030, the system captures a second one of the image frames, resulting in a captured image frame. The system may determine to capture, for example, all of the image frames that form a part of a video feed, in which case this act of capturing an image frame represents one particular image capture in a series of image captures.

At 1040, the system determines that the captured image frame is contaminated. The system may make this determination by transferring the captured image frame to an AI model #1 (for segmentation). The AI model #1 may then segment the part of the captured image frame that is obstructed.

At 1050, the system identifies a contaminated image segment from the captured image frame that is contaminated. The contaminated image segment may be sent to an AI model #2 (for generation) that is responsible for restoring the contaminated part. In other words, the system may input the contaminated image segment into the AI model #2 to preprocess the contaminated image segment for restoration.

At 1060, the system generates a prediction for a replacement image segment to replace the contaminated image segment. The system may use the AI model #2 to generate the prediction.

At 1070, the system composes the replacement image segment onto the cached image frame, thereby generating a composed image. The cached image frame may be, for example, the “last clear frame”. The system may use the AI model #2 to create a more faithful restoration using the “last clear frame”.

At 1080, the system replaces the captured image frame that is contaminated with the composed image.

Although the method 1000 has been described in a particular order with particular steps, it should be appreciated that the method 1000 may be carried out in a different order as described above, with fewer steps (e.g., with one of the steps omitted), or with more steps (e.g., with additional or intervening steps). For example, the method 1000 may end after step 1070 with the system generating a composed image (a) without continuing to step 1080 or (b) continuing to another postprocessing step.

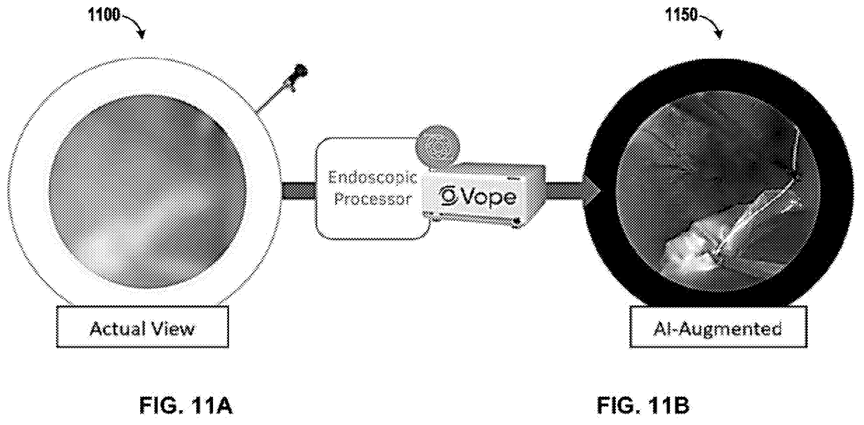

FIGS. 11A-11B show a first example of an actual surgical view and an improved view after image quality improvement. In FIG. 11A, the actual view 1100 shows the actual view from an endoscope before image quality improvement. In FIG. 11B, the improved view 1150 shows the improved view after image quality improvement. This example shows how the improved view 1150 provides an improvement over the actual view 1100 as a result of any one of the methods described herein, such as method 700, method 900, or method 1000. Advantageously, the improvement may, for example, be done in real time to assist during surgery. Also, advantageously, the inventors have observed a reduction in scope removals of up to 40% resulting from such image quality improvement.

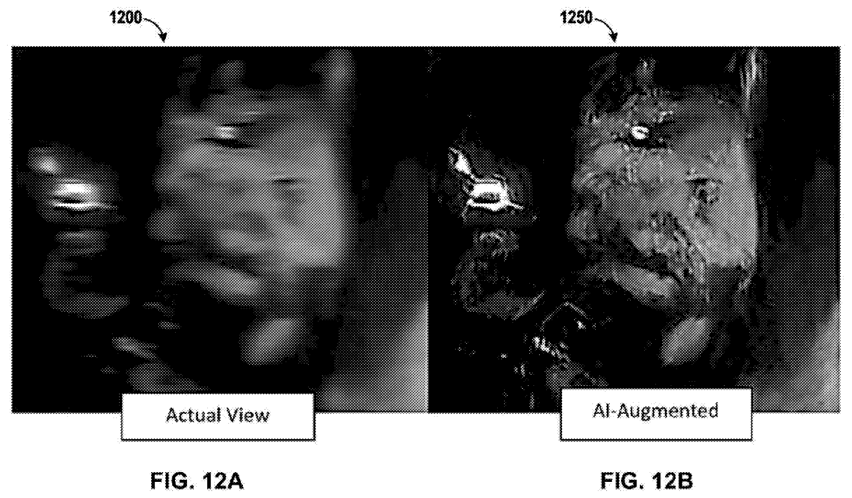

FIGS. 12A-12B show a second example of an actual surgical view and an improved view after image quality improvement. In FIG. 12A, the actual view 1200 shows the actual view from an endoscope before image quality improvement. In FIG. 12B, the improved view 1250 shows the improved view after image quality improvement. This example shows how the improved view 1250 provides an improvement over the actual view 1200 as a result of any one of the methods described herein, such as method 700, method 900, or method 1000. Advantageously, the improvement may, for example, be done in real time to assist during surgery.

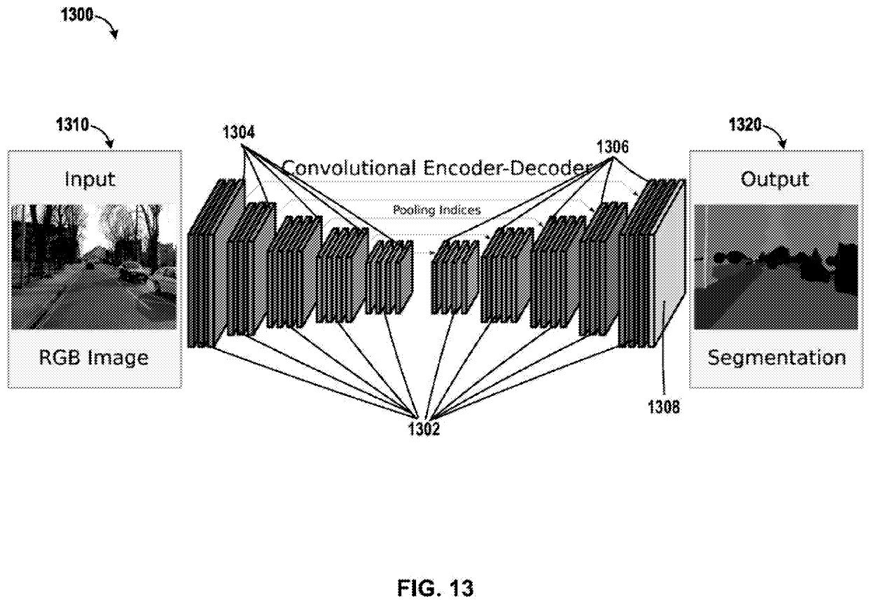

FIG. 13 shows an example of an architecture 1300 for image segmentation and restoration. In the architecture 1300, an input 1310 (e.g., an RGB image) is fed into a convolutional encoder-decoder. The convolutional encoder-decoder includes: convolution and batch normalization plus ReLU 1302; pooling 1304; upsampling 1306; and softmax 1308. The convolutional encoder-decoder then provides an output 1320 with the intended segmentation. This architecture 1300 may be a SegNet architecture. As such, there are no fully connected layers and it ends up being only convolutional. In particular, a decoder may upsample input using transferred pool indices from its encoder to produce one or more sparse feature maps. This may be followed by convolution with a trainable filter bank to densify the feature map. The final decoder output feature maps may then be fed to a softmax classifier for pixel-wise classification.

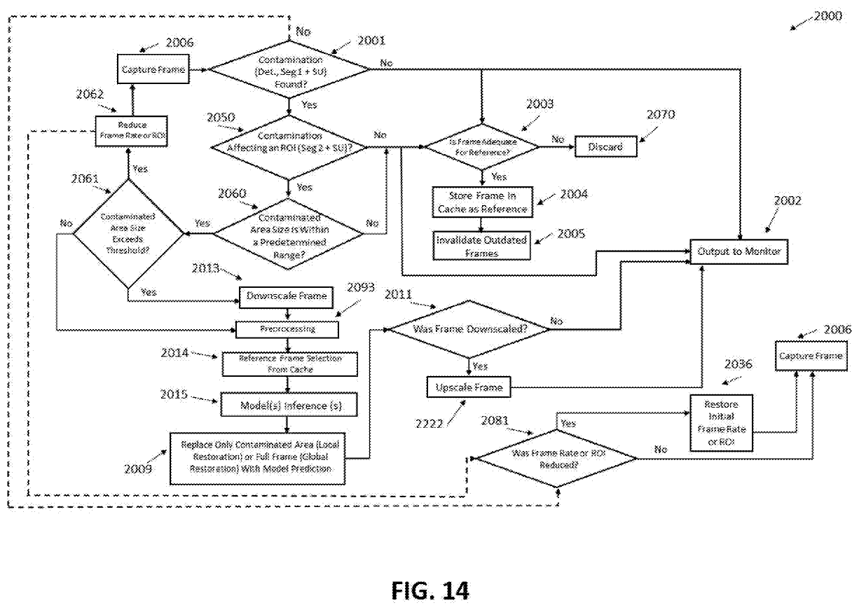

FIG. 14 illustrates a flow diagram of an exemplary embodiment of an image enhancement process 2000 for improving visual clarity through an endoscope. The process 2000 may be accomplished using some or all of the system 430, some or all of the system 210, or some or all of the system 100.

FIG. 14 illustrates a flow diagram of an exemplary embodiment of an image enhancement process 2000 for improving visual clarity through an endoscope. The process 2000 may be accomplished using some or all of the system 430, some or all of the system 210, or some or all of the system 100.

The process 2000 begins with capturing an image frame from an endoscopic video feed. In the process 2000, the system decodes the video signal (which may come, for example, from a DVI/SDI/HDMI interface) and converts it into individual frames at the same rate (e.g., at 30 FPS) to be processed by the system at step 2006.

After capturing the frame, the system performs contamination detection and segmentation in step 2001. Here, the system assesses whether any contaminants, such as fog, smoke, blood, fat, or blur, are present in the frame. These contaminants may originate in a variety of ways. For instance, while performing cauterization during surgical procedures, smoke could be generated due to the thermal interaction between the cauterizing instrument and biological tissue, such as blood vessels or muscle. This smoke may condense on the lens of the endoscopic camera or in the surgical room, generating a fog that obstructs the view of the camera. Furthermore, bleeding from incisions or vessel ruptures may cause blood to splash on the lens of the endoscopic camera. Additionally, manipulation of adipose tissue during surgical procedure may cause fat deposits to smear the lens. All these contaminations would obscure or blur the view of the camera. Moreover, sudden movement of the camera may cause a temporary loss of focus, resulting in a blurry image.

In some embodiments, instead of performing contamination detection and segmentation on a single frame, the system may process a group of frames collectively. Extracting information from multiple frames can provide a more comprehensive understanding of the scene and the contaminant, improving the accuracy of the image processing model or algorithm in detecting and segmenting contaminated regions. In some embodiments, based on the surgical step, available computational resources, or user input, the system may be configured to perform contamination detection and segmentation only on a subset of the group of frames.

During contamination detection and segmentation in step 2001, the system may use various approaches to identify the type and potential cause of contamination. Scene understanding (SU) may be employed to provide contextual awareness of the surgical scene and classify contamination type, aiding in subsequent processing steps. In some embodiments, machine learning models may be used, where the system processes the input frame to detect and classify contaminants automatically. For example, a machine learning model could take the frame as input and output the type of contaminant, such as smoke or blood. The machine learning model may include convolutional neural networks CNN(s), Transformers (for example, Vision Transformers) etc. In some embodiments, in order to reduce latency, models may be optimized using quantization (e.g., INT8 or FP16 precision) to reduce computational load, pruning to eliminate redundant weights, and knowledge distillation to create smaller, faster models while retaining accuracy. Deployment optimization frameworks like TensorRT, ONNX Runtime, or OpenVINO may be used to reduce inference latency. Techniques such as frame skipping and parallel processing on GPUs or another computing platform may also enhance throughput and reduce latency in some embodiments.

In some embodiments, machine learning models, such as CNNs with architectures like Faster Region-based CNN (R-CNN) may be used to localize contaminants such as fog, smoke, and blood by identifying and predicting bounding boxes around affected regions in an image. These models may be trained on annotated datasets where the contaminants are marked with bounding box coordinates, allowing the network to learn spatial patterns and object boundaries. Transformer-based localization models, such as Detection Transformers (DETR), may be used to enhance this process by capturing global context alongside local features, possibly improving the precision and robustness of localization tasks. Training may involve optimizing loss functions that combine classification loss (e.g., cross-entropy) with localization loss (e.g., smooth L1 loss), allowing for accurate bounding box predictions.

In some embodiments, machine learning models, such as CNN with architectures such as U-Net, may be used to segment contaminants. These models may be trained on annotated datasets, where each pixel is labeled according to the contaminant class, allowing the network to learn intricate spatial patterns. Transformer-based segmentation models with architecture such as Vision Transformers (ViTs), may be used to extend this capability by capturing global context in addition to local features. Training may involve optimizing loss functions such as the Dice coefficient or cross-entropy loss, allowing for precise boundary segmentation.

In some embodiments, traditional computer vision algorithms may be used. For instance, the system may analyze image histograms or other pixel-level attributes to detect contaminants based on distinct patterns, such as the darker areas associated with smoke or the bright, reflective spots caused by blood splashes.

In some embodiments, methods like edge detection with Sobel or Canny filters may be used to highlight boundaries where contaminants blur the image, while thresholding techniques may be used to segment regions based on intensity or color values. For example, foggy areas can be segmented by applying luminance thresholds, and blood regions can be detected using color space analysis in the HSV or YUV domains.

In some embodiments, a hybrid approach may be employed, combining machine learning and traditional algorithms for enhanced accuracy. By integrating machine learning models with traditional methods, the system may also cross-verify contaminant detection results or refine the detection accuracy.

A hybrid approach can leverage the strengths of both machine learning models and traditional algorithms to achieve efficient and accurate image analysis. Traditional methods, such as edge detection, thresholding, or histogram-based preprocessing, can quickly identify rough regions of interest in an image. Based on this preprocessing, the system can dynamically decide between applying localization or segmentation models in real time. For example, if the preprocessing suggests a well-defined region, a localization model like Faster R-CNN can be used to place bounding boxes around the contaminants for efficient processing. Conversely, if the contaminant boundaries are diffuse or complex, a segmentation model like U-Net can be applied to achieve pixel-level accuracy. This dynamic selection may balance computational efficiency with analytical precision such that the appropriate level of detail is provided while optimizing resource usage to reduce latency.

If no contamination is detected in the frame, the system proceeds to output the frame to the monitor in step 2002, allowing a clear view of the surgical field without additional processing. Furthermore, in step 2003, the system determines whether the frame is suitable for storage in cache, using predefined criteria. If the frame meets the necessary conditions, it may be stored as a reference for future restoration processes. The suitability assessment may involve calculating edge gradients across the frame, where higher gradient values indicate clearer, well-defined edges. Additionally, texture consistency may be analyzed by measuring local variations in pixel intensity, ensuring that fine anatomical details, like tissue texture and vascular patterns, are preserved. The suitability assessment may also involve parameters calculated based on as Dark Channel Prior (DCP), FADE (Fog Aware Density Evaluation) etc. These parameters may be utilized to quantify haze levels and the extent of obscuration. Similarly, entropy may be analyzed to measure the richness of visual information within the frame. A frame may be considered adequate for storing as a reference if it maintains a satisfactory level of visibility, even if it is mildly contaminated by artifacts such as light fog.

In some embodiments, a frame compromised by motion blur may be identified using techniques like the Variance of the Laplacian, which measures the sharpness of an image. A frame with significant blur may be discarded. In some embodiments, if the frame is detected as being out of the patient, it may be discarded. Additionally, if a surgical tool is detected too close to the lens (as determined by the area size of the image), the frame may be discarded as may obstruct too much of the scene, providing inadequate information for further processing.

A frame that meets one or more predefined thresholds for these metrics may be stored by the system in the cache as reference frames in step 2004. If a frame fails to meet one or more of these predefined thresholds, it is discarded, as indicated in step 2070. In some embodiments, the thresholds may be defined by the user, while in other embodiments, the system may be configured to automatically determine these thresholds based on predefined settings or real-time factors based on procedural needs or specific surgical requirements, etc.

Although the flow chart in FIG. 14 illustrates the caching of only uncontaminated sharp frames, i.e., those frames which require no restoration, in some embodiments, the system may also cache a frame that has undergone partial restoration, where only the unaffected, unprocessed areas are retained as reference frames. This approach enables the use of partially processed frames as reference without introducing AI-altered data into the restoration process, thereby maintaining fidelity in future restorations.

In the context of the present technology, the cache serves as a repository of selected frames that act as reference images to aid in restoring future frames that may be contaminated. By maintaining a set of recent, clear, and contextually relevant frames, the cache enables the system to apply accurate visual information to contaminated regions, thereby enhancing the quality of the video output.

In some embodiments, the cache could be implemented using high-speed Random Access Memory (RAM) to allow rapid access to reference frames. In some embodiments, for more extensive storage requirements, a dedicated Solid-State Drive (SSD) may be used, enabling both quick access and higher capacity for storing multiple frames.

The system may manage the cache by invalidating frames that are outdated or no longer relevant, as indicated in step 2005. Cache management helps in maintaining a set of reference frames that are temporally or contextually relevant to the current frame.

Temporally relevant frames are those frames which have been captured within a recent timeframe, such that they reflect the surgical environment and camera position in the recent past. In some embodiments, the timeframe for which a frame would be deemed as temporally relevant may be defined by the user of the system. In some embodiments, the timeframe for which a frame would be deemed as temporally relevant may be determined automatically by the system, based on a variety of factors, such as the speed of camera movement, the frequency of frame contamination, the specific procedural needs or specific surgical requirements, etc.

Contextually relevant frames are those frames that exhibit at least a certain degree of structural similarity to the current frame, closely matching its visual content and scene dynamics. Such frames are suitable for guiding restoration by providing reference data that aligns with the ongoing procedure and the current field of view. In some embodiments, the minimum degree of structural similarity that must be met by a frame to be deemed as contextually relevant may be defined by the user of the system. In some embodiments, the minimum degree of structural similarity that must be met by a frame to be deemed as contextually relevant may be determined automatically by the system, based on a variety of factors, such as the speed of camera movement, the types of detected contaminants, the complexity of the anatomical structures, the extent of the region affected by contamination, etc.

For cache management, the system determines which frames should be invalidated and removed from the cache, based on their temporal or contextual relevance to the current frame.

In some embodiments, the system may invalidate frames based on elapsed time, as frames older than a timeframe may no longer accurately represent the current surgical view.

In some embodiments, the system may invalidate frames based on contextual relevance of the frames. In this approach, structural similarity is evaluated between each cached frame and the current frame. A low structural similarity of a past frame compared to the current frame indicates that the camera is currently viewing a different region, meaning the past frame would not serve as an effective reference for the current frame.

The system may utilize the cached frames in a variety of ways. In some embodiments, both the current frame and a cached reference frame may be used during restoration, combining information from all regions of the two frames for a more comprehensive restoration process. In some embodiments, the system may selectively input only the contaminated region from the cached frame, rather than the entire frame, as a reference. This approach minimizes the processing load on the system, but may reduce the available visual context.

In some embodiments, the system may implement a mask-based approach, in which the current frame, a cached reference frame, and designated masks may be used during restoration. The masks serve as indicators, specifying which regions require restoration. The uncontaminated visual data from the cached reference frame may be used by the system to restore only the designated contaminated regions specified by the masks, ensuring that restoration is performed on affected areas while leaving uncontaminated regions unchanged. In some embodiments, the masks may be designated by the user, while in other embodiments, the masks may be automatically generated by the system based on detected contamination patterns or predefined settings.

Furthermore, for the restoration of a current frame, the system may select the appropriate reference frame from the plurality of cached frames in a variety of ways. In some embodiments, the system may select the immediate past frame as the reference frame. For example, if the surgical scene has remained stable and there is no significant camera movement between frames, the immediate past frame can provide a highly relevant reference for restoration, leveraging both temporal and contextual similarity.

In some embodiments, the system may select a non-immediate past frame as the reference frame. For example, in a scenario where the camera moves from position 1 at time t1 to position 2 at time t2, and then returns to position 1 at a later time t3, the system may retrieve an uncontaminated, sharp frame cached at time t1 as reference for restoring a contaminated frame captured at time t3, rather than using an uncontaminated, sharp frame cached at time t2. This approach avoids using a frame cached at position 2 and time t2, which, while being the immediate past frame, may not accurately match the current view. This selective retrieval allows the system to use a reference frame that better matches the current position and view, thereby enhancing restoration accuracy.

If contamination is detected in step 2001, the system proceeds to step 2050 to determine if the contamination affects at least one region of interest (ROI) in the frame, using a segmentation model alongside SU. In some embodiments, the at least one ROI may be defined by the user, using gestures such as zooming in on a region, or using a pointing device to select the region, etc. In some embodiments, the system may be configured to automatically determine the at least one ROI in the frame based on its knowledge of the current surgical procedure.

If the contamination does not affect any ROI, the system may bypass any restoration and output the frame directly to the monitor in step 2002. Furthermore, if the frame meets the criteria for caching, the system may store the frame in cache, as indicated in steps 2003 and 2004. In this case, although the frame is contaminated, it can still serve as a reference for restoring a contaminated ROI in a future frame, as the contamination does not impact any ROI.

However, if the contamination affects an ROI, the system advances to step 2060 to assess the size of the contaminated region.

In step 2060, the system evaluates whether the size of the contaminated region falls within a predefined range, considering factors such as contamination type and source. For example, if the contamination is dense fog, covering the entire field of view of the camera, the size of the contaminated region may exceed an upper limit, indicating that restoration may not be feasible. Conversely, if the contamination is minimal, such as a tiny, localized blood spot, the size of the contaminated region may fall below a lower limit, in which case the contamination can be neglected and no restoration is required. However, if the size of the contamination region is neither negligible nor too large to make restoration infeasible, the system may proceed to step 2061 to determine whether downscaling 2013, frame rate reduction or ROI size reduction 2062, or a combination of these adjustments are necessary to maintain near real-time operation. Depending on the specific embodiment, the system may trigger one or more of these adjustments.

In the context of the present technology, near real-time operation may be defined as minimizing perceived latency to a level where any delay between frame capture and display remains imperceptible to the human eye. End-to-end latency, which includes capture, processing, and display, may be maintained below 200 milliseconds to ensure near real-time operation. This may ensure that the surgical workflow remains uninterrupted, providing surgeons with a continuous and responsive video feed. The system may achieve this by dynamically adjusting processing parameters, such as resolution, frame rate, and ROI size, to balance computational efficiency and visual clarity while ensuring that restored frames are displayed with minimal delay.

In some embodiments, rather than downscaling the entire frame, only the ROI or the contaminated region within the ROI may be downscaled, which can reduce processing load while preserving important details in unaffected areas. In certain embodiments, this decision may be made by the user based on procedural needs, while in others, the system may automatically determine whether to downscale the entire frame or only the ROI or regions within the ROI, depending on factors such as the extent and location of the contamination.

Downscaling may be helpful when dealing with large contaminated areas because it reduces the image resolution, thereby decreasing the amount of data that the system needs to process. This reduction lowers computational load and latency, allowing restoration models to operate more efficiently and in less time, which is important in real-time applications like endoscopy. By processing a lower-resolution version of the image, the system can apply restoration models more quickly, minimizing latency without compromising essential visual information. For instance, a frame at 4K resolution (3840×2160) may require 50 milliseconds for restoration, whereas downscaling it to 1080 p (1920×1080) may reduce the required processing time to 20 milliseconds, ensuring that the restoration is completed within the time duration available before the capturing of the next frame, allowing near real-time operation.

In some embodiments, ROI size reduction may be used as an alternative to or in conjunction with downscaling and frame-rate reduction. The system may dynamically define a region of interest (ROI) within the frame, focusing processing efforts only on the most relevant areas, such as regions containing surgical tools or critical anatomical structures. The system may track these regions in real time, ensuring that image clarification is applied where it is most needed. By restricting restoration to a smaller, dynamically adjusted window rather than the entire frame, the system may reduce processing load, allowing faster restoration and ensuring real-time operation. For example, processing an entire 1080p frame may require 20 milliseconds, while limiting the processing to a 50% cropped ROI (960×540) could reduce the time to 10 milliseconds. Since restoring a smaller ROI requires fewer computations than processing the entire frame, this adjustment can further reduce latency, and allow near real-time operation.

In some embodiments, the system may dynamically adjust the frame rate of the video feed based on the computational resources available to the system, both in terms of software and hardware. In scenarios where restoration computations require more time per frame, the system may lower the frame rate to allow adequate processing time, ensuring that each restored frame is displayed before the next frame is captured.

For example, if the system is processing a 30 FPS video feed, each frame must be restored within 33.3 milliseconds to maintain real-time operation. If the system estimates that the time required for the restoration operations would exceed this timeframe, the system may reduce the frame rate to 20 FPS or lower, effectively increasing the available processing time per frame while maintaining smooth visualization. This adaptive frame rate adjustment may prevent processing delays and may reduce latency between captured and displayed frames, allowing near real-time operation.

Following downscaling at step 2013, or if it is determined in step 2061 that none of the adjustments (downscaling, frame-rate reduction, ROI size reduction) are necessary, the image undergoes preprocessing with respect to scene understanding in step 2093. In this step, the system applies adjustments to the frame based on the detected contaminant type, location, and the context of the surgical scene, thereby optimizing the frame for restoration in subsequent processing steps. For example, if the contamination is identified as fog, the system may apply contrast enhancements to increase the distinctions between different anatomical structures and improve overall visibility. If the contamination is identified as smoke, color corrections may be implemented to counteract any tinting effects caused by the dark smoke particles, restoring the color balance in the frame. For blood contamination, the system may employ brightness adjustments to reduce glare and prevent blood from overwhelming other details in the frame. Scene understanding may further inform these preprocessing adjustments by considering the specific ROI within the frame and the stage of the procedure. This tailored preprocessing step prepares the frame for model inference in the next step, enhancing restoration accuracy by ensuring that the most relevant visual information is optimized.

After preprocessing in step 2093, the system proceeds to reference frame selection from cache in step 2014. In this step, the system may retrieve one or more cached reference frames based on temporal relevance and contextual similarity to the current frame. The selection process may prioritize reference frames that exhibit high structural similarity to the contaminated frame while maintaining close temporal proximity to ensure alignment with the ongoing surgical scene. If multiple cached frames are available, the system may use predefined selection criteria, such as highest sharpness score to prioritize reference frames with the best clarity, closest temporal match to select frames captured in the most recent past, anatomical consistency to ensure that the reference frame represents the same anatomical structures as the current contaminated frame.

Next, the system proceeds to model inference in step 2015, utilizing both cached reference frames and scene understanding to guide the restoration. Here, a Mixture of Experts (MoE) approach may be used by the system, in which specialized models are selected based on the type and characteristics of the detected contamination. For instance, if fog has been identified, a defogging model may be activated to remove the fog and enhance contrast. In cases of motion blur, a deblurring model can be applied to sharpen details lost due to camera movement, while a despeckling model may be used to clear particulate noise caused by smoke. If blood or fat deposits are present, models designed to detect and mask these obstructions can be applied to reduce their visibility. In some embodiments, a combination of multiple models may be used if multiple contaminant types are present, such as applying both deblurring and despeckling models to address simultaneous motion blur and smoke contamination, ensuring restoration across multiple forms of contamination.

During the model inference in step 2015, Scene Understanding (SU) may assist in identifying key anatomical structures within the frame, thereby assisting in prioritizing cached frames that best align with the current scene context. For example, in a laparoscopic procedure targeting the gallbladder, if the current frame has a blurry or obscured view of an important anatomical feature, such as the cystic duct, SU can help the system retrieve a sharp, uncontaminated reference frame from the cache that shows the cystic duct clearly. This reference frame provides detailed visual information that aids in reconstructing the partially obscured regions in the current frame, enhancing clarity and continuity. By leveraging recent, contamination-free views of the cystic duct and surrounding structures, guided by SU, the system can generate a clear visualization of the anatomical features, helping the user to avoid accidental injury during dissection or isolation of the gallbladder.

In some embodiments, various machine learning architectures may be used for inference, including CNNs with solutions like U-Net or Transformers, such as Vision Transformers (ViTs), to capture global and local contexts. Generative Adversarial Networks (GANs), like Pix2Pix, may be effective for image-to-image translation, transforming contaminated images into clear ones. Inpainting models, such as LaMa, may be used for restoring missing or corrupted regions in an image. These architectures rely on large, annotated datasets encompassing diverse contamination scenarios to ensure robust performance in surgical environments.

In some embodiments, data augmentation techniques, such as adding synthetic smoke, fog, or blur, may be used to further improve model generalization. Tailored loss functions, including mean squared error for deblurring or perceptual loss for GANs, may be employed to optimize training. Evaluation metrics like Peak Signal-to-Noise Ratio (PSNR), Structural Similarity Index Measure (SSIM), and mean absolute error may be used to assess fidelity and similarity to ground truth.

Next, in step 2009, the system determines whether to apply the model inference for local restoration or global restoration, depending on the extent and distribution of the contamination. Local restoration may be performed when the contamination affects only a portion of the frame, particularly if the contamination is confined to one or more regions of interest (ROIs), such as areas where surgical tools or critical anatomical structures are located. In such cases, the system may restore only the contaminated region while preserving the unaffected portions of the frame, causing no alteration to uncontaminated areas. Conversely, global restoration may be applied when the contamination is widespread across the frame, making localized restoration insufficient to maintain visual clarity. For example, if fog or smoke diffuses uniformly across the scene, or if motion blur affects the entire frame due to sudden camera movement, the system may opt for full-frame restoration to correct the entire frame.

In some embodiments, the system may automatically select between local and global restoration based on contamination segmentation results. In other embodiments, the system may allow the user to manually select the restoration approach based on procedural needs, such as prioritizing local restoration for preserving the clarity of unaffected surgical regions or opting for global restoration when comprehensive image enhancement is required.

Once the restoration process is completed, the system may evaluate whether the frame was downscaled earlier in step 2013. If the frame was downscaled, the system proceeds to step 2222, where the restored frame undergoes upscaling to restore its original resolution. In some embodiments, the upscaling may be performed using interpolation-based techniques such as bilinear or bicubic interpolation. In some embodiments, a category of machine learning models known as super-resolution models may be used to upscale the image, restoring the details in the original frame.

In some embodiments, the resolution level needed may depend on the specific anatomy under view, as some surgical tasks may require higher resolution to provide a detailed view of smaller or more intricate anatomical structures, such as in pediatric cases or when viewing small anatomical regions, for example, fine blood vessels or delicate nerve pathways.

In some embodiments, the user may be given the option to choose whether upscaling is required for a particular frame or a particular region of a frame, using gestures such as zooming in on a section of interest, or using a pointing device to select the region, etc. In other embodiments, the system may be configured to automatically determine when upscaling is needed, based on factors such as the type of anatomical structure detected, specific surgical need, the type of contaminants detected, the extent of the contaminated region, etc.

Additionally, in step 2081, the system may determine whether contamination was detected at step 2001 or whether the frame rate or ROI size was previously reduced in step 2062. If a contamination was detected, or a reduction of ROI frame size was made, the system may proceed to step 2036, where the original frame rate or ROI size is reinstated.

Once the frame has been restored through model inference and if needed, upscaled, it may undergo further refinement in post-processing. During post-processing, the system may apply distinct visual cues or labels to inform the user about areas that have undergone AI-based restoration. These cues or labels may provide information regarding the specific regions modified by the AI, the types of contaminants detected, the restoration models used, and the associated confidence levels for each restoration.

For instance, the system may overlay a subtle, semi-transparent color code to highlight areas restored by different models, such as a blue tint for regions processed with a defogging model, a green tint for areas processed with deblurring, or a red tint for areas restored from cached frames. A brief, unobtrusive label may appear near the restored region, displaying details such as ‘AI: Fog Removal’ or ‘AI: Deblurring Applied,’ along with a confidence percentage, such as ‘Confidence: 95%,’ to reassure the user of the reliability of the restored frame.

In some embodiments, the system may further provide an interactive feature that allows the operator to toggle AI-restored regions on or off temporarily. This comparison tool enables the operator to see the original contaminated view alongside the restored version, facilitating greater trust in the AI-generated enhancements.

Additionally, in some embodiments, small icon indicators, such as a glowing dot or dashed outline around processed regions, may be used by the system to alert the user in real time whenever AI-based restoration has been activated in a specific area. These visual markers can be fine-tuned for transparency, color, and size, such that they are easily noticeable without obstructing the user's view of essential anatomical structures.

It is contemplated that these visual cues and labels may be valuable during delicate surgical procedures, where real-time information on restoration processes enables the user to make informed decisions. By incorporating these detailed indicators, some embodiments of the present technology offer options for verification of the reliability of AI-restored frames and greater user control.

It is contemplated that in various embodiments of the present technology, one or more image processing operations in the process 2000, including contamination detection, segmentation, restoration, and enhancement, may be performed using machine learning models, traditional computer vision algorithms (e.g., histogram analysis, edge detection etc.), or a combination of both.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A method for near real-time image enhancement of endoscopic images, the method executable by at least one processor, the method comprising:
accessing a first image from a stream of images;
determining that a contaminated segment is present within the first image;
determining at least one of (i) a type of contaminant associated with the contaminated segment or (ii) a dimension of the contaminated segment;
adjusting at least one of (iii) a resolution of images from the stream of images or (iv) a frame rate associated with the stream of images, the adjusting being based on the at least one of (v) the type of contaminant or (vi) the dimension of the contaminated segment, the adjusting resulting in at least one of (vii) generating reduced-resolution images from the stream of images or (viii) a reduced stream of images;
inputting, to a first machine-learning algorithm (MLA), (ix) at least one reduced-resolution image from the reduced-resolution images or (x) at least one extracted image taken from the reduced stream of images to generate a replacement segment for the contaminated segment; and
generating a restored image based on the replacement segment.

2. The method of claim 1, wherein the contamination type is indicative of at least one of smoke, fog, blood, fat, blur, or presence of a surgical tool.

3. The method of claim 1, further comprising:
accessing a second image from a cache memory storing a plurality of cached images, the accessing the second image from the cache memory comprising:
determining at least one of:
a temporal relevance score of the plurality of cached images, the temporal relevance score being indicative of a time elapsed between capturing an individual cached image and the first image; or
a contextual relevance score of the plurality of cached images, the contextual relevance score being indicative of a degree of structural similarity between an individual cached image and the first image; and
selecting, from the plurality of cached images, the second image based on the temporal relevance score meeting a temporal relevance threshold or the contextual relevance score meeting a contextual relevance threshold.

4. The method of claim 3, further comprising:
determining whether the first image is adequate for caching, the adequacy being determined based on at least one image quality metric; and
in response to determining that the first image is adequate for caching, storing the first image in the cache memory as one of the plurality of cached images.

5. The method of claim 4, wherein the at least one image quality metric comprises one or more of:
a sharpness metric, determined based on at least one of an edge gradient value and a variance of Laplacian; or
a haze level metric, determined based on at least one of a Dark Channel Prior (DCP) value and a Fog Aware Density Evaluation (FADE) value; or
an information richness metric, determined based on entropy analysis.

6. The method of claim 5, wherein an individual cached image is removed from the cache memory in response to at least one of:
the temporal relevance score of the individual cached image failing to exceed the temporal relevance threshold; and
the contextual relevance score of the individual cached image failing to exceed the contextual relevance threshold.

7. The method of claim 1, further comprising selectively upscaling the restored image.

8. The method of claim 1, further comprising selectively increasing the frame rate associated with the stream of images.

9. The method of claim 1, further comprising selectively adjusting a dimension of a region of interest (ROI) used to determine that the contaminated segment is present within the first image.

10. The method of claim 1, further comprising:
generating a visual cue on the restored image, the visual cue indicating at least one of:
a region corresponding to the replacement segment; and
a reliability score for the replacement segment within the restored image.

11. The method of claim 1, wherein determining that the contaminated segment is present within the first image comprises executing a second machine-learning algorithm (MLA) by inputting the first image to the second MLA and outputting, by the second MLA, at least one of (i) a type of contaminant associated with the contaminated segment or (ii) a dimension of the contaminated segment.

12. The method of claim 1, wherein prior to determining that the contaminated segment is present within the first image, the method executes grouping the first image with another image from the stream of images.

13. The method of claim 1, wherein determining that the contaminated segment is present is executed on some images but not all images of the stream of images.

14. The method of claim 1, wherein adjusting at least one of (iii) the resolution of images from the stream of images or (iv) the frame rate associated with the stream of images is based on available computing resources of a device on which the method is executed.

15. The method of claim 1, wherein prior to inputting to the first MLA, (ix) the at least one reduced-resolution image from the reduced-resolution images or (x) the at least one extracted image taken from the reduced stream of images, the method executes:
selecting the first MLA from a plurality of MLAs based on at least one of:
the type of contaminant associated with the contaminated segment; or
available computing resources of a device on which the method is executed.

16. The method of claim 1, wherein the at least one extracted image taken from the reduced stream of images is further processed to reduce its resolution before being used to generate the replacement segment for the contaminated segment.

17-32. (canceled)

33. A system for near real-time image enhancement of endoscopic images, the system comprising at least one processor configured to:
access a first image from a stream of images;
determine that a contaminated segment is present within the first image;
determine at least one of (i) a type of contaminant associated with the contaminated segment or (ii) a dimension of the contaminated segment;
adjust at least one of (iii) a resolution of images from the stream of images or (iv) a frame rate associated with the stream of images, the adjusting being based on the at least one of (v) the type of contaminant or (vi) the dimension of the contaminated segment, the adjusting resulting in at least one of (vii) generating reduced-resolution images from the stream of images or (viii) a reduced stream of images;
input, to a first machine-learning algorithm (MLA), (ix) at least one reduced-resolution image from the reduced-resolution images or (x) at least one extracted image taken from the reduced stream of images to generate a replacement segment for the contaminated segment; and
generate a restored image based on the replacement segment.

34. (canceled)

35. The system of claim 33, wherein the at least one processor is further configured to:
access a second image from a cache memory storing a plurality of cached images, the accessing the second image from the cache memory comprising:
determining at least one of:
a temporal relevance score of the plurality of cached images, the temporal relevance score being indicative of a time elapsed between capturing an individual cached image and the first image; or
a contextual relevance score of the plurality of cached images, the contextual relevance score being indicative of a degree of structural similarity between an individual cached image and the first image; and
selecting, from the plurality of cached images, the second image based on the temporal relevance score meeting a temporal relevance threshold or the contextual relevance score meeting a contextual relevance threshold.

36-48. (canceled)

49. The system of claim 33, further configured to display the restored image.

50-64. (canceled)

65. A non-transitory computer-readable medium comprising instructions, which upon being executed by at least one processor, configure the at least one processor to:
access a first image from a stream of images;
determine that a contaminated segment is present within the first image;
determine at least one of (i) a type of contaminant associated with the contaminated segment or (ii) a dimension of the contaminated segment;
adjust at least one of (iii) a resolution of images from the stream of images or (iv) a frame rate associated with the stream of images, the adjusting being based on the at least one of (v) the type of contaminant or (vi) the dimension of the contaminated segment, the adjusting resulting in at least one of (vii) generating reduced-resolution images from the stream of images or (viii) a reduced stream of images;
input, to a first machine-learning algorithm (MLA), (ix) at least one reduced-resolution image from the reduced-resolution images or (x) at least one extracted image taken from the reduced stream of images to generate a replacement segment for the contaminated segment; and
generate a restored image based on the replacement segment.

66-85. (canceled)
